(19) United States
(12) Patent Application Publication
(10) Pub. No.: US 2010/0143905 A1
(43) Pub. Date: Jun. 10, 2010

(54) METHODS AND COMPOSITIONS FOR MULTIVALENT BINDING AND METHODS FOR MANUFACTURE OF RAPID DIAGNOSTIC TESTS

(76) Inventors: Michael J. Lane, Baldwinsville, NY (US); Jerrie Gavalchin, Groton, NY (US); Brian D. Faldasz, Littleton, MA (US)

Correspondence Address: KLAUBER & JACKSON, 411 HACKENSACK AVENUE, HACKENSACK, NJ, 07601, US

(21) Appl. No.: 12/450,532

(22) PCT Filed: Mar. 28, 2008
(86) PCT No.: PCT/US2008/004100

§ 371 Date: Dec. 30, 2009

Related U.S. Application Data
(60) Provisional application No. 60/920,649, filed on Mar. 29, 2007.

(51) Int. Cl. C12Q 1/68

(52) U.S. Cl. 435/6

(57) ABSTRACT

The invention provides reagents and methods for multivalent binding and quantitative capture of components in a sample. In one aspect, reagents and methods for diagnostic assay for antigen, ligand, binding agent, or antibody are provided. Compositions of a non-natural or deliberately constructed nucleic acid-like polymeric scaffold are provided, to which multiple antibodies, peptides or other binding agents can be affixed by hybridization of a oligonucleotide: binding agent complex such that the nucleic acid: binding agent construction displays multivalent behavior when interacting with a multivalent analyte. Methods for constructing and using the scaffolds are described. Such compositions may include assembly of mixed specificity binding agents such that the composition displays multivalent binding behavior against a target containing mixed analytes which can be bound by the construct to effect a binding affinity increase such as is observed in avidity reagents against single analytes expressed multiply on the target analyte. A manufacturing method for producing rapid diagnostic assays in a decentralized manner is also described. The method generates net economic advantages over conventional diagnostic manufacturing practices.

### FIELD OF THE INVENTION

The present invention relates generally to reagents and methods for multivalent binding and quantitative capture of components in a sample. In one aspect, reagents and methods for diagnostic assay for antigen, ligand, binding agent, or antibody are provided. Compositions of a non-natural or deliberately constructed nucleic acid-like polymeric scaffold are provided, to which multiple antibodies, peptides or other binding agents can be affixed. A manufacturing method for producing rapid diagnostic assays in a decentralized manner is also described. The method generates net economic advantages over conventional diagnostic manufacturing practices.

### BACKGROUND OF THE INVENTION

### Multivalent Binding

It has been known for some time that the “apparent” affinity of a molecule for another can be improved if both reactants exhibit a “valency” for each other greater than 1:1 (c.f. P. J. Hogg and D. J. Winzor, (1985) “Effects of ligand multivalency in binding studies: a general counterpart of the Scatchard analysis.” Biochim. Biophys. Acta 843 159-163. This can be accomplished by “polymerizing” the reactants involved (c.f. Terskikh et al., 1997 PNAS 94: 1663-1668 “‘Peptabody’: a new type of high avidity binding protein”) which will alter the apparent equilibria versus the 1:1 situation.

To make this binding advantage more clear, consider that the strength of the interaction of polymerized antigen with polymerized antibody would involve multiple antibody:antigen interactions. Affinity refers to the strength of binding between a single antigenic determinant and an individual antibody combining site whereas avidity refers to the overall strength of binding between multivalent antigens and antibodies. Avidity is a measure of the overall strength of binding of an antigen with many antigenic determinants and multivalent antibodies. Avidity is influenced by both the valence of the antibody and the valence of the antigen and is more than the sum of the individual affinities. The factors contributing to avidity are complicated. Consider the extreme case of an antigen with ten thousand, 10⁴, antigens on the cell surface interacting with an antibody polymerized so as to produce one hundred, 10² physically connected antibodies. The increased valency of both antigen and antibody will lead to a decrease in overall dissociation rate, versus that exhibited by the individual reagents interacting, just from the perspective that the probability that all antibody antigen interactions will dissociate simultaneously is exceedingly small. One can view this as a dissociation rate argument, i.e. if one interaction is dissociated, the others will remain associated, thus enhancing the probability that dissociation of any particular antibody:antigen interaction will be unlikely to cause complete dissociation of the entire reactant: analyte complex. The apparent dissociation rate of the complex involving equilibria between multiple “functionally connected” antibodies and antigens (i.e. each species connected together physically in a manner that yields multivalent behavior) will effectively approach zero as the degree of multivalency is increased (c.f. Hubble, J., 1999) although the precise approach to effective zero dissociation and the actual dissociation rate reduction realizable under the conditions of the experiment (with increasing multivalency) will differ from reaction to reaction.

Classically, chemical reactions, especially biochemical reactions, are conceptualized or designed from the standpoint of singular components interacting to form products. Such interactions are generally described in terms of binding, and binding reactions characterized as singular molecules of each species joining as reactants. In addition, it is well known in the art that concentrations of the reactants are essential quantities in describing the reactions, and, in fact, in the creation of products from such reactions. Researchers have noted that aggregations of one of the reactants can dramatically influence these reactions from the standpoint of the characteristics of critical binding events in the reactions if the target analyte of the reactant is also multivalent. This added binding due to multiple copies of the reactant coupled together is known as avidity. Avidity is a term that describes the interaction between multivalent substances. One example of the avidity capture strategy of the present invention for human CD4 cells is shown in FIG. 1B. Assuming that any CD4+ cells bound by the capture reagent can be detected, the present invention increases the apparent ‘affinity’ of the anti-CD45 antibody by employing it in a polyvalent construction. This is in contrast to the usual antibody:antigen capture approach, shown in FIG. 1A. In effect, we are exploiting the polyvalency displayed by the CD45 receptor on the cell surface by allowing these receptors to bind to our polyvalent anti-CD45 constructs. This will increase the valency of the CD45 and anti-CD45 interaction which will lead to a “bonus” binding effect due to cooperativity of the association and dissociation of the observed binding reaction (versus monovalent binding to the receptor). In other words, the probability that all anti-CD45 antibody interactions will dissociate simultaneously becomes exceedingly small as the number of anti-CD45:CD45 interactions increases, if the anti-CD45 antibodies are linked together (c.f. Hubble, 1997, Minga et al., 2000). One antibody dissociating from a single receptor will not cause the complex to dissociate. In addition, the spatial localization of any dissociated antibody: antigen complex enhances the probability that any particular dissociated interaction will re-associate more quickly than when the reactants are free in solution. In effect, the overall dissociation rate will approach zero at some level of anti-CD45 antibody “chaining”.

In general (i.e. as depicted in FIG. 1A), the interaction of anti-CD45 (the “capture reagent”) with a single receptor can be described by the standard free energy relationship for two interacting species, e.g.

ΔG=−RT ln Ka (1)

where ΔG is Gibbs free energy, R is the gas constant, T is the absolute reaction temperature, and Ka is the association rate constant for the two species.

However, since CD45 is a ‘polyvalent’ receptor on T cells (it is expressed as multiple copies), if we make the capture reagent polyvalent for the CD45 receptor (e.g., by coupling anti-CD4 antibodies together using a linear polymer) we would have the requisite parameters for an avidity capture reagent where the free energy governing the reaction becomes:

ΔG_{avidity}=Σ₁^{|n-m|}f(ΔG) (2)

or, in terms of the equilibria involved

K_{avidity}=Π₁^{|n-m|}(Ka) (3)

where: n=number of anti-CD45 antibodies in avidity construct, m=number of CD45 receptors available for binding, and f is an adjustable parameter describing the apparent increase In observed binding reaction per additional anti-CD45.

Of course, this effectively statistical description, while retaining the expected relationship from the interaction of two polyvalent species interacting, does not take into account the “geometry” of the binding elements (CD45 receptor and anti-CD45 antibody). The CD45 receptor could appear in dense clusters on the cell surface or be dispersed sparsely or display some combination of these extremes across the surface (e.g., dense clusters sparsely distributed). However, from a purely statistical description, and assuming that there are no steric issues, we can expect that with as few as 10 anti-CD45 interactions from any given coupled anti-CD45 avidity construct it would be unlikely that the interaction could be displaced by monovalent anti-CD45 at any reasonable concentration (Hubble et al., 1995; Hubble, 1997; Daniak et al., 2006).

These constructed aggregations of reactant molecules are typically organized in some fashion, as in dendrimers where a number of reactants are held together by chemical “tethers” in a branching, tree-like structure. All such modifications share the design intention of improving the binding of the reactants to one another by virtue of multiple binding interactions (reviewed in Gestwicki et al., 2002).

In theory, binding reactions are described in terms of equilibrium equations, which provide mathematical models for the overall behavior of a reaction. Any given equilibrium can be manipulated toward forming product by known approaches such as LeChatelier's principle. However, in practice, ELISA reactions which are designed to detect as small an amount of analyte as possible are practically constrained by factors such as limits on the amount of capture antibody bound and noise introduced by the detector step. In practice, in vivo delivery of drug moieties is also limited by the concentrations of potential pharmaceuticals that can be administered without either toxicity or disadvantageous immune responses in the organism. Similarly, in vivo delivery of vaccine formulations has the same toxicity and disadvantageous immune response issues but also is recognized to need exercise of control over the observed effective response of the immune system.

Increased binding affinity for specific target molecules is a desired characteristic of reagents of value to a broad range of industries, including pharmaceutical, molecular diagnostics, chemical purification and decontamination, and water and waste treatment. However, the design of reagents with enhanced binding affinity is nontrivial. Various approaches to increasing the binding constant of a reagent have been proposed, many of which are very effective. Too high a binding constant, however, can actually result in loss of overall specificity, as non-target molecules of similar composition become targets as well. The key advantage of the present invention is that it maintains the specificity of a desirable binding agent while effectively decreasing the overall dissociation rate of the reactants. The ability to bind specific targets with both specificity and slow dissociation rate permits specific trapping of molecules for further processing, e.g., extracting disease-indicating targets for later detection or for purification purposes, interfering with the ability of receptors to function due to steric occlusion, and/or removal of dilute targets for either disposal in a concentrated form or for further use of the purified target. Target molecules for such purposes may include metals, toxins, cells, viruses, and complex synthetic and/or naturally occurring molecules.

### Manufacturing of Diagnostic Tests

A conventional (e.g. first world) manufacturing and distribution model for rapid diagnostic test manufacture and development involves a centralized manufacturing facility where components are assembled. Assembled components are then distributed from the central location. The need for up-front acquisition of expensive manufacturing equipment to manufacture such assays can create a formidable barrier to assay deployment, particularly in remote locations or in instances or regions where price and cost is a significant factor. Further, there is still a need and advantage for a highly efficient and low cost on site diagnostic manufacturing capacity, even in field applications or in doctors' offices or critical care facilities. To address these issues, we propose a rapid diagnostic assay-manufacturing model in which a liquid deposition device, for instance a low-cost inkjet printer, is employed to “print” such assays with components either obtained from a quality controlled central source or locally manufactured.

Therefore, in view of the aforementioned deficiencies attendant with prior art assays and methods of manufacturing assays, it should be apparent that there still exists a need in the art for simple, rapid, highly sensitive, and low cost binding assays as well a method to manufacture these assays quickly, at low cost and potentially on site.

The citation of references herein shall not be construed as an admission that such is prior art to the present invention.

### BRIEF DESCRIPTION OF THE DRAWINGS

FIGS. 1A and B depicts standard and multivalent capture assays. A) Depicted is a T cell “sandwiched” between a capture anti-CD45 antibody and a detector anti-CD4:AP conjugate attached to a surface such as a microwell as in an ELISA assay. B) Modification of the capture and detector antibodies to present as polyvalent “avidity” binders to increase the apparent Kd. It is not necessary that the detector antibody also be multiplexed to execute the instant invention.

FIG. 2 depicts an isothermal signal amplification scheme on inkjet printed nitrocellulose. Schematic depiction of experimental approach demonstrating quantitation by assembly and analysis of a tetravalent DNA scaffold construct. In this example, twelve lines of streptavidin: AP were printed at a predetermined concentration (suspended in TBS (tris-buffered saline)) sufficient to generate a low intensity signal after 15 min exposure to BCIP/NBT color generator. On a 3 mm wide “strip” this represents 1.5×10⁹ copies per line for a total of 18×10⁹ copies per strip. The strips were then blocked for 20 min. in 0.5% casein. Next a total of 2.5×10¹² copies of 5′ biotin; d(T)25 in 100 ul TBS was allowed to flow up the “test” strip while 100 ul TBS alone was allowed to flow up the “control” strip. Both strips were then subjected to a 100 ul TBS wash step followed by a poly d(A) “flow” step wherein a total of 9×10⁹ copies of polyd(A) in 100 ul TBS (conversion 1 OD=23 ug (see www.genosphere-biotech.com/custdna/tech_corner.htm) were allowed to flow up both strips. Following another 100 ul TBS wash step, 100 ul of pre-equilibrated streptavidin:AP+5′ biotin; d(T)25 (at a molar ratio of 1.0 to 0.8, respectively) in TBS was allowed to flow across both the test and control strips (total copy number ˜8.8×10¹² molecules streptavidin:AP) followed by another 100 ul TBS wash step and a 15 min immersion in BCIP/NBT developer (after removal of the “wicking” pad). The reaction was “fixed” by immersion of the strips in a 10 ug/ul solution of proteinase K in TBS.

FIG. 3 depicts the results of amplified versus control obtained from the experiment described in FIG. 2 above. The strips are shown at the left of the diagram in both normal and expanded view. Scans of the strips obtained employing an HP flatbed scanner are shown on the upper right. [Note: All signal intensities were within the linear response range as determined in previous calibration experiments-data not shown.] At the bottom of FIG. 3 are plots of the signal intensities (peak height determined from the bitmap image using Scion Image Beta 4.0.3; Scion Corporation). In this case, the signal intensity increase over background was approximately four-fold, consistent with the reported size of the polyd(A) chain length of 125-150 bases (25 base biotin:d(T)25 anchor to membrane bound streptavidin plus four additional biotin:d(t)25:streptavidin:AP molecules, i.e. a total of 5 d(T)25 molecules per polyd(A)).

FIGS. 4 A and B. A) depicts an InkJet Printer and ink cartridge employed in the antibody and analyte printing experiments. B) provides assembly steps for inkjet printed lateral flow assay. 1) Millipore lateral flow card stock was cut to desired size (i.e. depending on number of test strips desired), taped to 8.5×11 in. paper and antibody (or other protein) printed. Printing involved opening an HP27 print cartridge, removing the black ink and foam followed by rinsing extensively with water. Then the “screen” over the printhead was removed carefully with tweezers. The print cartridge was then extensively rinsed again with water followed by printing distilled water continuously over an entire page to “purge” the printhead of any remaining ink residue. Then 200-250 microliters of antibody/protein solution was added (spiked with yellow food dye to monitor printing). Any pattern may be constructed in a graphics package (we used Microsoft Powerpoint). After printing, the cartridge was rinsed with water and purged by printing a page with distilled water. It can be used repeatedly if washed appropriately. 2) The printed card stock was then cut into 3 mm “strips” after removing the plastic from the “wick” side of the cut strip. 3) a “wicking pad” was attached such that it overlaps the nitrocellulose by ˜2-3 mm. To conduct the test, one simply places the tip of the strip into a 100 microliter solution placed in a “flat bottom” container and allows the solution to “flow” across the nitrocellulose and be absorbed by the wick pad.

FIG. 5 depicts the CD4 Dipstick Design. The test is set out as a “dipstick” style test that requires that the “stick” be dipped into a diluted whole blood container (screw-cap vial), or any sample solution, whereupon the cells will then flow up the membrane (e.g. nitrocellulose) with the T-cells adhering to the printed avidity capture reagent. The key features are a series of four identical anti-CD2 T cell capture lines (pre-titered to effect capture of 10⁴ cells/line) followed by a gap and a “test worked” line (composed of printed recombinant CD4) at a concentration sufficient to produce color with the anti-CD4 avidity detection reagent as it flows across the membrane.

FIGS. 6A and B. A) Components of a standard lateral flow assay. The assay is shown side-on in order to illustrate the features of the assay. The entire assay is mounted in a plastic housing with an orifice for sample addition over the sample pad. Once sample is added, it “flows” through the conjugate pad where detector (e.g. AP coupled antibody for colorometric detection, nanogold coupled antibody, quantum dot, etc) binds the analyte of interest); then sample flows up the nitrocellulose membrane where analyte is bound at the reagent lines; sample continues to “flow” and crosses the “test worked line” generating color and a successful assay. B) Modifications we have adopted for the CD4 assay. Of note is there is no plastic housing and the assay is a simple dipstick which is held by the operator and dipped successively into: 1—blood sample vial, 2—rinse and blocking reagent vial, 3—avidity labeling reagent vial and 4—BCIP/NBT color generator. See FIG. 5 above for a 3D view and size specifications.

FIG. 7 provides an initial antibody printing result. Goat anti-IgG HRP conjugate was printed onto plastic-backed Azon inkjet media (i.e. flow card stock paper). Antibody conjugate was suspended in 250 ul of 10 mM Tris buffered saline at a concentration of 10 ng/uL and the solution was “spiked” with 10 uL yellow food dye to monitor printing. Antibody solution was placed in HP27 inkjet cartridge after rinsing out the black ink solution. Color development was allowed to proceed for ˜1.0 min. at RT in 1 mL of substrate solution contained in a 1.5 mL polypropylene tube, and stopped by rinsing with ddH2O. Green color is a “hybrid” of yellow food dye (tracking dye) and blue HRP product. Pattern was generated in Microsoft Powerpoint.

FIG. 8 provides a graph showing hypothetical concentrations of mycobacteria and antibodies through stages of Mycobacterium avium subsp. paratuberculosis (Map) infection. Horizontal line suggests test detection level.

FIGS. 9 A and B. A) Background reduction with the use of a “blocking step” in which a 100 uL solution of TBS and casein is allowed to “wick up” a nitrocellulose membrane prior to exposing to detector antibody and color development. Note the reduction in background “noise” in 0.5 percent casein versus 0.25 percent. (Note that without any blocking agent step background approached signal—not shown) (B) Actual test strip before (1) and after (2) detection steps. On the left is an assembled strip with wicking pad (8 strips of Whatman 3 mM paper, capacity ˜700 uL) to facilitate flow of reagent vertically up the membrane. Strip was preprinted with biotinylated goat IgG at 3.2 ug/uL in TBS. The strip was first placed in a flat bottom vessel containing 200 uL TBS+0.5% casein. After that fluid was depleted, the strip was moved to a vessel containing 200 uL TBS+0.05 ug/uL streptavidin:AP conjugate, followed by a 100 uL wash step in TBS. Total time for these steps is currently 45 minutes. The strip was allowed to dry and then immersed in BCIP/NBT for ten minutes. The reaction was stopped by immersing the strip in 1 mL dd H2O. The strip was then scanned on an HP flatbed scanner.

FIG. 10 depicts the Map antibody Lateral Flow Assay Design. In essence, the test is a “dipstick” test that requires that the “stick” be dipped into a diluted whole blood or serum, or other sample solution. The blood, serum or solution will then flow up the nitrocellulose membrane with the Map antibody adhering to the printed avidity (Map antigen) capture reagent. The key features are a series of four identical anti-Map antibody capture lines, followed by a gap and a “test worked” line.

FIGS. 11A and B. A) Depicted is a standard assay of anti-Map antibody “sandwiched” between a capture Map antigen and a detector anti-bovine Ig:AP conjugate. B) Modification of both the capture and detector to present as polyvalent “avidity” binders to increase the apparent Kd.

FIGS. 12A, B, and C depicts DNA avidity constructs. A) As a control, conjugated Map antigen construct is employed as a monovalent anti-Map antibody binder. B) We have also designed two different complimentary oligonucleotides, both of which are 5′ tailed with dT25. C) The dT25 sections of these oligonucleotides will allow assembly onto polyd(A)n, if desired.

FIG. 13 depicts T cells captured via anti-CD2 antibody and detected with biotin: anti-CD4. The first lane shows nitrocellulose card stock after printing anti-CD2 antibody. T cells (Jurkat T lymphoma cells ATCC TIB-152), pretreated with anti-CD4 antibody were wicked across the membrane and bound to the anti-CD2 capture antibody. Poly d(A) solution was wicked up the membrane to convert the bound anti-CD4 to a polyvalent configuration. After applying FITC d(T)20 conjugate, signal was visualized using anti-FITC:alkaline phosphatase. The processed test strip with positive signal is shown.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Cells, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular hybridization reaction. Deming appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA which are degenerate to those set out herein. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC |
| | or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

Mutations can be made in nucleic acid sequences such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein.

The following is one example of various groupings of amino acids:

Amino acids with nonpolar R groups—Alanine, Valine, Leucine, Isoleucine, Proline, Phenylalanine, Tryptophan, Methionine
Amino acids with uncharged polar R groups—Glycine, Serine, Threonine, Cysteine, Tyrosine, Asparagine, Glutamine
Amino acids with charged polar R groups (negatively charged at Ph 6.0)—Aspartic acid, Glutamic acid
Basic amino acids (positively charged at pH 6.0)—Lysine, Arginine, Histidine (at pH 6.0)
Another grouping may be those amino acids with phenyl groups: Phenylalanine, Tryptophan, Tyrosine
Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions.

The present invention should be considered to include amino acid sequences containing conservative changes which do not significantly alter the activity or binding characteristics of the resulting polypeptide, antigen or antibody. Similarly the nucleic acid sequences set out herein are exemplary and should not be interpreted as limiting. Therefore, changes, alterations, additions and deletions can be made in the sequences to alter length, G-C content, extent of hybridization, length of homologous or hybridizing nucleic acid, percent identity, degree of homology, etc.

A “heterologous” region of the nucleic acid construct is an identifiable segment of nucleic acid within a larger nucleic acid molecule that is not found in association with the larger molecule in nature. Thus, when the heterologous region encodes a mammalian gene or portion thereof, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” can include an immunoglobulin, including antibodies and fragments thereof, that binds a specific epitope. The term encompasses polyclonal, monoclonal, single chain, Fv, fragments, and chimeric antibodies, the last mentioned described in further detail in U.S. Pat. Nos. 4,816,397 and 4,816,567.

An “antibody combining site” is that structural portion of an antibody molecule comprised of heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptan with a reagent such as iodoacetamide. An antibody containing intact antibody molecules, or containing the combining site, is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. An antibody may be constructed of a plurality of antibody combining sites, each immunospecific for a different antigen; e.g., a bispecific (chimeric) monoclonal antibody.

The general methodology for making monoclonal antibodies by hybridoma technology is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890.

Methods for producing polyclonal anti-polypeptide antibodies are, well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference.

The phrase “pharmaceutically acceptable” refers to molecular entities and compositions that are physiologically tolerable and do not typically produce an allergic or similar untoward reaction, such as gastric upset, dizziness and the like, when administered to a human.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The present invention relates generally to reagents and methods for multivalent binding of components in a sample. The invention further relates to reagents and methods for quantitative capture of components in a sample. In one aspect, reagents and methods for diagnostic assay for cells, antigen, ligand, binding agent, or antibody are provided. The reagents include polymeric scaffolds for binding of components in a sample. The scaffolds may be composed or comprised of nucleic acid and/or polypeptide. Exemplary compositions of a non-natural or deliberately constructed nucleic acid-like polymeric scaffold are provided, to which multiple antibodies, peptides or other binding agents can be affixed.

The invention provides a system for the capture of at least one analyte of interest in a sample, said system comprising:

(A) a substrate or solid support which is a wickable medium suitable for the reception and transport of said sample;
(B) a scaffold or polymer having a repeating unit, which scaffold or polymer is bound covalently or non covalently to the substrate or support of (A);
(C) a first capture reagent capable of binding directly or indirectly with analyte in the sample, which first reagent is affixed to or interspersed with the scaffold or polymer of (B);
(D) optionally a second capture reagent or binder, capable of binding (i) to both said first capture reagent and to an analyte in the sample or (ii) to a second analyte in the sample, which second reagent is affixed to or interspersed with the scaffold of (B) or which binds covalently or non covalently to the first capture reagent of (C);
(E) an indicator means which indicates that the sample has been transported along the substrate or support and confirms that the reagent(s) are operable.

The first capture reagent may comprise one or more component or capture reagent. The second capture reagent may comprise one or more component or capture reagent. Additional capture reagents may be added so as to modify, enhance selectivity, specificity and/or signal and detection. In aspects of the system, the substrate or solid support is selected from glass, nylon, paper, nitrocellulose, and plastic; the scaffold or polymer is selected from nucleic acid, peptide, carbohydrate, and protein; the first capture reagent is selected from antibody, antigen, peptide, nucleic acid, protein, ligand, carbohydrate, metal, fat, oil, and organic compound; the second capture reagent or binder is selected from antibody, antigen, peptide, nucleic acid, protein, ligand, carbohydrate, metal, fat, oil, and organic compound. The indicator means may be a predetermined amount of analyte.

In a further embodiment, the system further comprises a detector for quantifiable detection of analyte in the sample. The detector may be selected from a label, radioactive element, enzyme, or dye. In an embodiment, the detector is covalently attached to the first or the second capture reagent. In various aspects, the detector comprises an antibody, antigen, ligand, peptide, protein, nucleic acid or carbohydrate which binds or otherwise interacts with the analyte.

The invention provides a test kit for quantitation of one or more antibody or antigen in a sample comprising:

(A) a substrate or solid support which is a wickable medium suitable for the reception and transport of said sample and which is selected from glass, nylon, paper, nitrocellulose, and plastic;
(B) a scaffold or polymer having a repeating unit, which scaffold or polymer is bound covalently or non covalently to the substrate or support of (A) and which is selected from nucleic acid, peptide, carbohydrate, and protein;
(C) a first capture reagent capable of binding directly or indirectly with the antibody or antigen in the sample, which first reagent is affixed to or interspersed with the scaffold or polymer of (B) and which is selected from antibody, antigen, peptide, nucleic acid, protein, ligand, carbohydrate, and organic compound;
(D) optionally a second capture reagent or binder, capable of binding (i) to both said first capture reagent and to an antibody or antigen in the sample or (ii) to a second antibody or antigen in the sample, which second reagent is affixed to or interspersed with the scaffold of (B) or which binds covalently or non covalently to the first capture reagent of (C);
(E) an indicator means which indicates that the sample has been transported along the substrate or support and confirms that the reagents are operable, wherein the indicator is a predetermined amount of analyte; and
(F) a detector for quantifiable detection of antibody or antigen in the sample which detector is selected from a label, radioactive element, enzyme, or dye.

This invention also provides a manufacturing method for producing rapid diagnostic assays in a decentralized manner and at low cost. The method generates net economic advantages over conventional diagnostic manufacturing practices. The methods and compositions of this invention provide a means for producing and conducting rapid and sensitive assays on site in poor, remote, low technology, or high throughput locations or situations.

The invention provides a method for the manufacture of an analyte capture strip to be used for capture of at least one analyte in a sample, which strip comprises

(A) a substrate or solid support which is a wickable medium suitable for the reception and transport of said sample, wherein the substrate is a printable medium;
(B) a scaffold or polymer having a repeating unit, which scaffold or polymer is bound covalently or non covalently to the substrate or support of (A);
(C) a first capture reagent capable of binding directly or indirectly with analyte in the sample, which first reagent is affixed to or interspersed with the scaffold or polymer of (B);
(F) optionally a second capture reagent or binder, capable of binding (i) to both said first capture reagent and to an analyte in the sample or (ii) to a second analyte in the sample, which second reagent is affixed to or interspersed with the scaffold of (B) or which binds covalently or non covalently to the first capture reagent of (C);
(G) an indicator means which indicates that the sample has been transported along the substrate or support and confirms that the analyte of interest has been captured; comprising selecting a liquid deposition device and depositing each or any of the scaffold, first capture reagent, second capture reagent, and indicator with said liquid deposition device in a regular and predetermined pattern. In one embodiment, the liquid deposition device is an inkjet printer.

The invention provides a process for application of a liquid reagent to a printable surface for capture of an analyte in a sample, said process utilizing an inkjet printer, comprising loading the liquid reagent into a printer ink cartridge for said inkjet printer and printing the reagent in a regular and predetermined pattern on the printable surface.

Specific and effective binding of an agent or receptor to a target or ligand is important if not essential to the activity and function in various aspects of physiology, biology, diagnostics, drug development, purification and component analysis. Antibodies function via recognition and binding to their antigens or epitopes. Ligands function via recognition and binding to their receptors. Drug companies often assay for new agents by testing and screening for activity based on recognition and binding to a preselected target. Similarly, diagnostic assays include a binding requirement, often in both the selection and detection aspects of an assay. This invention utilizes binding chemistry, kinetics and capacity to provide rapid and sensitive assay systems.

Numerous drug compounds function by binding to receptors, targets or sites, including antigens or antigen binding sites. High avidity compounds offer the advantage of potentially lower required dosage, with corresponding lowered risk of adverse effects. Targets for such compounds include host cell receptors, viral coat protein domains, bacterial cell receptors, polypeptide active sites. In each of these cases, binding of the drug compound results in the inability of the pathogen to consummate its function of interacting with and corrupting host cell function.

This invention provides reagents and methods for production of vaccines. The reagents include polymeric scaffolds for binding of antigen, that would result in slow release and persistence of antigen, both of which are desirable in a vaccine. In addition, the scaffold could function as an adjuvant, in a manner similar to current DNA vaccines or CpG adjuvants.

Specific binding of target molecules with high avidity is of tremendous importance for effective molecular diagnostics. The ability to bind and hold targets from a relatively dilute sample (e.g., blood sample), permits concentration of these dilute targets which enables the use of detection methods that have previously only been useful for targets present in high concentrations in the sample (e.g., alkaline phosphatase and other color-generating chemistries). The cost advantages of such approaches enables high volume applications (e.g., point-of-care assays) that would otherwise be prohibitively expensive in both specialized equipment and highly-trained personnel for operation and correct interpretation of results of same. Examples include both detection and quantification of specific cell types, cancer cells, viral load, bacterial infection, biotoxins and other foreign protein targets, and inherent markers of host disease conditions (e.g., diabetes, genetic markers, various cancers, adverse cardiovascular conditions).

Purification and/or identification of specific cell populations such as in diagnostics, monitoring, for transplantation or other therapeutic applications offers yet another application for the present invention. High avidity binding agents, e.g., constructs of the present invention bound to a filter membrane, can allow for the extraction of desired cell populations, from blood, bone marrow or spinal fluid, for example. In a similar application, undesirable cells or proteins could be removed from the blood; for example, leukemic cells, could be removed prior to autologous bone marrow transplantation of a leukemia patient.

Requirements for detection and identification of bioterrorism, chemical warfare and explosive agents are similar to those of the most sensitive diagnostic applications. Target molecules can be expected to be highly dilute in the sample (water, air). In this application, the need for field-testing is even greater than for point-of-care diagnostics. The characteristics of the present invention enable trapping of extremely dilute target molecules for further detection or analysis.

In bioremediation, extraction of some undesirable or environmentally damaging or toxic molecules from groundwater and/or wastewater is currently both expensive and time consuming. The present invention enables more efficient and higher throughput removal of contaminants than conventional approaches by, e.g., using membranes, surfaces or filters that have been coated with the polyvalent binding constructs of the present invention and thereby obtaining a higher capture/filter efficiency at potentially higher flow volumes.

Purification of drinking water offers yet another application for the present invention. High avidity binding agents, e.g., constructs of the present invention bound to a filter membrane, can allow for the extraction of various biological and chemical molecules from the water.

The chemical and biotechnology industries routinely require extraction and concentration of molecular species to obtain pure reagents. This application of the present invention is, in effect, the reverse of the water purification application, where the molecules captured from the solution can then be further concentrated and purified.

Testing for or purification/extraction of chemical contaminants at low levels, for example the detection of antibiotics in milk and soil, pesticides and industrial pollutants in water and soil, could also be accomplished with the present invention. Veterinary applications, including but not limited to diagnostics, pharmaceuticals and vaccines, are similar to those already described for human medical applications.

Testing for contaminants and infectious agents in meat and produce can be accomplished with the present invention, offering higher sensitivity to targets than presently available rapid tests due to the high avidity characteristics of the present invention. Targets captured for these purposes can then be further processed, e.g., as for diagnostic applications.

The present invention is particularly applicable in remote locations and in epidemic or chronic disease situations. For instance, it would be useful in malaria-infected parts of the world for rapid, cost-effective diagnosis and assessment. In situations where there is potentially epidemic or disease, the assay and methods provide rapid, accurate and cost-effective assessment and monitoring, enabling critical treatment to those in need.

In the descriptions that follow, the term “antibody” refers generally to any of a variety of molecules that specifically recognize and bind preferentially to one chemical or molecular species. It is clear to one skilled in the art that, in addition to biological antibodies or immunoglobulins as noted above, also included in the term “antibody” as used herein are peptides, polypeptides, proteins, and other molecular moieties having the capability of preferential recognition and binding to particular molecular species. Further and similarly, the term “antigen” refers generally to any of a variety of binders or molecules that are recognizable as distinct entities or families of entities by an antibody (as defined above), and can include peptides, nucleic acids, metals, carbohydrates, fats, oils, etc.

In general, the composition of present invention includes a polymer, called here a “scaffold”, to which is affixed more than one antibody or antigen. In one instance, the polymer scaffold is a single stranded nucleic acid molecule such as a PNA, DNA, RNA, etc. or a double stranded nucleic acid molecule or even a triplex DNA molecule to which antibody or binder is bound through the coupling of the antibody to an oligonucleotide of sequence composition suitable to bind at multiple sites along the scaffold. The presence of multiple antibodies in close proximity results in the higher avidity of the construct to an antigen or antigens, which antigens are themselves of multivalent structure, than a single antibody would demonstrate. In the alternative where the antigen is not of classic multivalent structure (i.e. multiple copies of the same antigenic “site” on the same target molecule) the oligonucleotides are attached to different antibodies (polyclonal antibody against the antigen, for example) with differing recognition sites on the antigen so as to effect multivalency of the interaction. The scaffold may be, but is not necessarily, bound, covalently or non-covalently, to a solid support such as glass, nylon, paper, nitrocellulose, plastic, etc.

In a particular embodiment, a deoxyribonucleic acid polymer of known sequence is used to provide a scaffold to which multiple antibodies are attached to generate a polyvalent composition. Since a property of nucleic acids is to “hybridize” with complementary sequences to form a duplex, a preferred method for attaching the antibodies to the scaffold is to employ hybridization of complementary oligonucleotides. Of course, in the case of “hybridizing” an oligonucleotide to a duplex nucleic acid, the oligonucleotide is designed so as to form “triplexes” at various sites along the linear duplex nucleic acid using knowledge of triplex recognition rules (c.f. Gowers and Fox, 1999). In this approach, the antibodies are attached, (for instance, chemically, enzymatically, or by other means known in the art), to an oligonucleotide “backbone” that is comprised of a sequence complementary to the scaffold sequence or a portion thereof. The capture molecule:backbone complexes are then hybridized to the scaffold. Capture molecules may be spaced evenly or unevenly along the length of the scaffold depending on the initial sequence design and the complementary sequences attached to the antibodies.

In a particular embodiment, the number of antibodies attached to the backbone polymer is two or greater.

In another particular embodiment, the scaffold includes one or more “synthetic” bases or modified bases, e.g., PNA or synthetic linkages between the bases such as thiophosphate, phosphorothioate linkages which are resistant to nucleases. In another particular embodiment, the scaffold construct is comprised of a two or more phosphodiester or phosphodiester-like linkages. Thus, the nucleic acid or oligonucleotide in the scaffold may comprise at least one nucleotide modified at the 2′ position of the sugar, most preferably a 2′-O-alkyl, 2′-O-alkyl-O-alkyl or 2′-fluoro-modified nucleotide. Such modifications are routinely incorporated into oligonucleotides and these oligonucleotides have been shown to have a higher Tm (i.e., higher target binding affinity) than 2′-deoxyoligonucleotides against a given target. In another preferred embodiment, the oligonucleotide is modified to enhance nuclease resistance. Nucleic acids which contain at least one phosphorothioate modification are particularly preferred for in vitro applications (Geary, R. S. et al (1997) Anticancer Drug Des 12:383-93; Henry, S. P. et al (1997) Anticancer Drug Des 12:395-408; Banerjee, D. (2001) Curr Opin Investig Drugs 2:574-80). Specific examples of some preferred oligonucleotides envisioned for this invention include those containing modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones. The amide backbones disclosed by De Mesmaeker et al. (1995) Acc. Chem. Res. 28:366-374) are also preferred. Also preferred are oligonucleotides having morpholino backbone structures (Summerton and Weller, U.S. Pat. No. 5,034,506). In other particular embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al., Science, 1991, 254, 1497). Nucleic acids may also contain one or more substituted sugar moieties. Oligonucleotides may comprise one of the following at the 2′ position: OH, SH, SCH₃, F, OCN, heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. Similar modifications may also be made at other positions on the oligonucleotide, particularly the 3′ position of the sugar on the 3′ terminal nucleotide and the 5′ position of 5′ terminal nucleotide. Nucleic acids may also include, additionally or alternatively base modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-me pyrimidines, particularly 5-methylcytosine (5-me-C) (Sanghvi, Y. S., in Crooke, S. T. and Lebleu, B., eds., Antisense Research and Applications, CRC Press, Boca Raton, 1993, pp. 276-278), 5-hydroxymethylcytosine (HMC), glycosyl HMC and gentobiosyl HMC, as well as synthetic nucleobases, including but not limited to, 2-aminoadenine, 2-thiouracil, 2-thiothymine, 5-bromouracil, 5-hydroxymethyluracil, 8-azaguanine, 7-deazaguanine (Kornberg, A., DNA Replication, W.H. Freeman & Co., San Francisco, 1980, pp 75-77; Gebeyehu, G., et al., 1987, Nucl. Acids Res. 15:4513). A “universal” base known in the art, e.g., inosine, may be included.

It is not necessary for all positions in a given nucleic acid or oligonucleotide to be uniformly modified, and more than one of the aforementioned modifications may be incorporated in a single oligonucleotide or even at a single nucleoside within an oligonucleotide.

In yet another particular embodiment, the scaffold construct phosphodiester linkage is coupled to a sugar in an alternating sugar pattern, wherein the alternating sugar phosphodiester backbone links a binding agent, where the binding agent may be selected from the group comprised of any of numerous known binding agents for a multivalent ligand. In this embodiment, metal ions, peptides, proteins, dyes, alkyl chains, chemical groups, etc. can provide the binding agent, and a minimum of two binding agents are linked to provide a multivalent binding affinity to the multivalent ligand, where any of metal ions, peptides, proteins, dyes, alkyl chains, etc. comprise the reactive sites of the multivalent ligand.

In another particular embodiment, two copies of an antibody against an antigen, which antigen contains at least two binding sites for the antibody, are linked to a scaffold of alternating composition of deoxy-ribose with the antibody attached, by any of a variety of methods known in the art, to the backbone by means of the ribose. For example, the antibody can be linked to a scaffold of alternating composition of deoxy-ribose with the antibody attached to a “base” as understood to be Adenine, Guanine, Cytidine, Thymine, uridine, etc where other bases are known to those in the art and can be chemically modified (by methods known in the art).

In a particular embodiment multitude (2, 3, 4, 5, 10, 20, 200, 2000 or more) of antibodies can be attached to the sugar-phosphodiester polymer “backbone”, such that a number of antibodies are attached to a single backbone.

In another particular embodiment polydeoxyribonucleic acid polymer of fixed sequence can be used to provide the “backbone” for multiple attached antibodies to generate a polyvalent composition. In this embodiment the two antibodies are attached either directly or indirectly (including for instance via biotin) to a single strand DNA sequence complementary to the “backbone” sequences in at least one position along the “backbone”.

Since a property of nucleic acids is to “hybridize” with complementary sequences to form a duplex, a single strand DNA of defined sequence is synthesized and the multivalent composition created via hybridization of complementary oligonucleotides to which an antibody has been attached by any of a variety of methods known in the art. Antibodies may be spaced evenly or unevenly along the length of the single strand DNA polymer depending on the initial sequence design and the complementary sequence attached to the antibody. In a preferred embodiment, the number of antibodies attached to the backbone polymer is two or greater. In another preferred embodiment, the sugar phosphodiester backbone polymer includes one or more “synthetic” bases, e.g., PNA.

In one such embodiment, the number of antibodies attached is two in tandem such that a “nicked polyvalent duplex” DNA is obtained. In this and further embodiments, an oligonucleotide of sequence A is synthesized (on a DNA synthesizer) so as to form a continuous DNA chain with an “A” sequence repeated twice. An oligonucleotide complementary to “A” is covalently coupled to an antibody through any of several methods (c.f. Schweitzer et al., (2000) “Immunoassays with rolling circle amplification” PNAS 97: 10113-101 19; supplementary material), which oligo-antibody conjugate is then incubated with the “A” sequence so as to form a duplex containing two copies of the oligo complex bound to the “A” sequence.

In another embodiment, the number of antibodies attached is two in tandem such that a “gapped polyvalent duplex” DNA is obtained. In this embodiment the oligo:antibody target complex is separated by a gap introduced into the target sequence of 1, 2, 3., etc. bases which do not hybridize with the oligo:antibody conjugate. Such an arrangement of the construct would be expected to allow more steric movement of the two antibodies when interacting with the target binding molecules as the flexibility of the backbone DNA molecule would be expected to increase.

In another embodiment, the number of antibodies attached is three in tandem such that a “nicked polyvalent duplex” DNA is obtained.

In another embodiment, the number of antibodies attached is three with single strand DNA between each of the duplexes formed such that a “gapped polyvalent duplex DNA” is obtained.

In another embodiment a long DNA polymer backbone is employed to hybridize tens to hundreds of oligonucleotide conjugated antibodies. These hybridizations can, by design, result in nicked or gapped polyvalent duplex DNA, and/or a mixing of the same.

In further aspect of the invention, the nucleic acid, for instance the sugar phosphodiester backbone polymer, is employed for dual purposes: first, as a backbone for the structure, and second, as a molecular recognition target for binding, as for linking the structure to a solid support. In a preferred embodiment, the attachment to the solid support will employ another sugar phosphodiester backbone polymer composed of complementary sequence to the recognition target sugar phosphodiester backbone sequence of the structure.

In another particular embodiment, the antibodies are attached to the backbone by means of sugar phosphodiester backbone hairpin structures. The hairpin structures may attach to the backbone by employing complementary (to the backbone) sequences at the open end of the hairpin, such that the two strands of sugar phosphodiester backbone comprising the open end of the hairpin form duplex with a portion of the backbone.

In another preferred embodiment, antibodies are attached to the backbone by chemical means, for example by the use of a heterobifunctional crosslinking agent such as SMCC (Pierce: Succinimidyl 4-[N-maleimidomethyl]cyclohexane-1-carboxylate) or sulfo-SMCC (Pierce: Sulfosuccinimidyl 4-N-maleimidomethyl cyclohexane-1-carboxylate) which compounds allow coupling of oligonucleotides to proteins covalently. Other such coupling chemistries can be used to effect covalent attachment of an oligonucleotide to a protein through either terminal base residues of the oligonucleotide or internal residues of the oligonucleotide.

In yet another embodiment, the reaction is comprised of: 1) a multivalent antibody constructed as described here and employed as a capture antibody construct; 2) an antigen, that is, a target molecule or cell of interest; and 3) a multivalent antibody constructed as described here and employed as a detection antibody construct. In this embodiment, the detection antibody construct has been further modified so as to provide a means for signaling its presence, e.g., by means of direct attachment of dye (visible, fluorescent, phosphorescent, etc.) molecules.

In another embodiment, the signaling means employs any of a variety of signal amplification methods and/or compositions, numerous examples of which are well known to those skilled in the art.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs) oligonucleotide conjugated to an antibody against a particular cell surface receptor such that binding affinity is increased over that displayed by a monovalent form of the antibody and the construct therefore serves as a better binder of the receptor(s) to which the antibody binds. Such a construct can then be employed to “capture” a particular multivalent analyte from solution which allows better measurement of the analyte at lower copy number than the monovalent form of the antibody. Here analyte may be a virus, cell, receptor, protein, peptide, drug, metabolic product, etc. Such constructs may be employed in ELISA, lateral flow, agglutination, or other diagnostic formats to aid in measurement of the particular analyte.

In a further aspect of the invention, the scaffold can be utilized as a therapeutic composition or in therapeutic applications. In a further aspect, the scaffold can be utilized as an in vivo diagnostic or imaging agent or an agent to deliver specific therapeutic substances (toxins, drugs, radionuclides) to cells (drug delivery).

In one such embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs) oligonucleotide conjugated to an antibody against a toxin such that binding affinity is increased over that displayed by a monovalent form of the antibody and the construct therefore serves as a better binder of the toxin. After in vivo delivery this results in “coating of the toxin” such that the toxin cannot effectively interact with its in vivo target.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs) oligonucleotide conjugated to an antibody against a particular contaminant such that binding affinity is increased over that displayed by a monovalent form of the antibody and the construct therefore serves as a better “agonist” of the receptor(s) to which the antibody binds after in vivo delivery. In this embodiment the construct may, preferably, be attached to a solid support or filter.

In another embodiment a linear polydA (or other defined sequence) molecule of 40, 60, 80, . . . bases in length is hybridized with a dT25 (or other sequence complementary to the first sequence) oligonucleotide conjugated to an antibody against a particular cell surface receptor such that binding affinity is increased over that displayed by a monovalent form of the antibody and the construct therefore serves as a better “agonist” of the receptor(s) to which the antibody binds after in vivo delivery.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs)) oligonucleotide conjugated to an antibody against a particular cell surface receptor such that binding affinity is increased over that displayed by a monovalent form of the antibody and the construct therefore serves as a better “antagonist” of the receptor(s) to which the antibody binds after in vivo delivery.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs)) oligonucleotide conjugated to an antibody against a particular cell surface receptor such that binding affinity is increased over that displayed by a monovalent form of the antibody and the construct therefore serves as a better binder of the receptor(s) to which the antibody binds after in vivo delivery. If, in addition to the antibody:dT25 conjugate, a drug:dT25 conjugate (where drug represents a peptide, a protein, an enzyme, an anti-tumor drug, etc.) was also incubated with the polydA such that both the antibody and drug dT25 conjugates are “statistically mixed” on the polydA backbone then the added antibody specificity for its receptor will enhance delivery of the drug to its target cell.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs)) oligonucleotide conjugated to an antibody against a particular cell surface receptor such that binding affinity is increased over that displayed by a monovalent form of the antibody and the construct therefore serves as a better “antagonist” of the receptor(s) to which the antibody binds after in vivo delivery.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs)) oligonucleotide conjugated to an antibody against a particular cell surface receptor such that binding affinity is increased over that displayed by a monovalent form of the antibody and the construct therefore serves as a simply better binder of the receptor(s) to which the antibody binds after in vivo delivery which results in apoptosis and subsequent cell death.

In another embodiment a linear polydA (or other defined sequence) molecule of 40, 60, 80, . . . bases in length is hybridized with a dT25 (or other sequence complementary to the first sequence where the length can be defined as 1, 2, 3, . . . n such that hybridization occurs)) oligonucleotide conjugated to an antibody against a viral cell surface protein such that binding affinity is increased over that displayed by a monovalent form of the antibody and the construct therefore serves as a simply better binder of the cell surface protein to which the antibody binds. After in vivo delivery this results in “coating the virus” such that the virus cannot effectively interact with its in vivo target.

In any of the above in vivo aspects, addition or incorporation of a label, radioactive element, enzyme or dye provides for imaging or detecting binding in vivo. The label may be selected from enzymes, ligands, chemicals which fluoresce, radioactive elements etc. In the instance where a radioactive label, such as the isotopes ³H, ¹⁴C, ³²P, ³⁵S, ³⁶Cl, ⁵¹Cr, ⁵⁷ Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

In a preferred embodiment, the scaffold is comprised of a single, repeating subunit (e.g., repeating DNA, PNA, RNA “bases”, e.g., poly-dA, poly-dT, poly-dG, poly-dC, poly-U).

In another preferred embodiment, the scaffold is comprised of different subunits, the sequences of which provide binding domains for the sugar phosphodiester backbone sequences, e.g, complementary sequences, in sufficient quantity to offer multiple binding domains (e.g., 2, 3, 4, . . . , 10, 20, . . . ) along the length of the scaffold.

In yet another preferred embodiment, multiple different sequences are employed on the same backbone scaffold, each of the different sequences bearing a different antibody with affinity for a different substrate (e.g., different cell receptor, different protein recognition site, etc.) and capable of hybridizing with at least one position along the backbone.

In another preferred embodiment, the scaffold is comprised of different subunits, the sequences of which provide binding domains for two or more nucleic acid, e.g. sugar phosphodiester backbone, sequences. These binding domains can be placed in the scaffold so as to effect a variety of patterns of binding for the sugar phosphodiester backbones. For example, given two sugar phosphodiester backbone sequences (“A” and “B”), binding patterns on the scaffold can organized to establish different orders of backbones, and therefore antibodies, along the scaffold, e.g., “AAAABBBB”, “ABABABAB”, “AABBAABB”, “AABBBBAA”. It is obvious to one skilled in the art that other variations of such sequences are possible and can be used. In addition, it is obvious to one skilled in the art that more than two different backbone sequences (e.g., 3, 4, 5, . . . , 10, 20, . . . ) can be employed for these constructs.

In yet another preferred embodiment, the scaffold is comprised of all or part of the sequences of a plasmid, e.g., pBR322, M13 or like constructs, which sequences are then employed as hybridization targets for backbone structures. Sequences of particular value in this embodiment are those that are repeated, e.g., 2, 3, 4, 5, 6 times throughout the overall sequence of the plasmid. A further embodiment employs the plasmid in combination with backbones of mixed sequences that are complementary to various sequences comprising the plasmid. This approach enables targeting of the backbones to specific, predetermined locations on the plasmid sequence, and enables different mixtures of backbone sequences to be employed for different purposes, e.g., attachment to solid support, attachment of antibodies, and the like.

In another preferred embodiment, the scaffold is comprised of a single, repeating subunit, and multiple sugar phosphodiester backbones to which different binding molecules are attached are allowed to “compete” for binding domains on the scaffold. The relative numbers of different binding molecules can be varied to any desired proportion of one to the others e.g., by varying the ratios of the different binding molecules borne by the sugar phosphodiester backbones introduced into the reaction.

In another preferred embodiment, the scaffold is comprised of subunits defining binding domains that are immediately adjacent to one another with respect to the scaffold. For example, in the case of scaffold and backbones composed of DNA, the resulting duplex would form a “nicked” duplex, with the nicks appearing between each of the backbones hybridized to the scaffold.

In another preferred embodiment, the scaffold is comprised of subunits defining binding domains that are “spaced” along the scaffold, e.g., binding domain sequences on the scaffold are interspersed between non-binding domain subunit sequences.

In another embodiment, the scaffold is affixed to a solid support by any of numerous means known in the art of attachment of a polymeric molecule to a solid support, including but not limited to, affinity binding, attachment of a binding molecule at one end of the scaffold, chemical binding, UV cross-linking, etc.

In another embodiment, the scaffold, and any molecules or structures bound to it, is permitted to remain in solution.

In another embodiment, the scaffold is of sufficient physical length to bridge between two distinct regions on a solid support. In this embodiment, molecular provisions are incorporated into the scaffold (e.g., by means known in the art, and/or by means described in the present invention), so that the scaffold binds to both the first and the second regions of the solid support. Note that, in this embodiment, the regions of the scaffold that are not involved with binding of the scaffold to the solid support are available for use as binding domains for sugar phosphodiester backbones, as provided for in the present invention.

In another preferred embodiment, the scaffold:sugar phosphodiester backbone complex is constructed prior to introduction of the analyte.

In another embodiment, the complex is built up, in a step-wise fashion, on a solid support, e.g., by first affixing the scaffold to the solid support, then binding the nucleic acid, e.g. sugar phosphodiester, backbones bearing the target analyte binding molecules, then introducing the sample containing the target analyte, etc. It is obvious to one skilled in the art that different orders of addition of components to the reaction will produce the same complexes.

In another embodiment, the complex is built up, in a step-wise fashion, in solution, e.g., by introducing the scaffold and the sugar phosphodiester backbones bearing the target analyte binding molecules into the reaction, and then introducing the sample containing the target analyte, etc. It is obvious to one skilled in the art that different orders of addition of components to the reaction will produce the same complexes.

In another embodiment, the complex is built in a single reaction, e.g., by creating a mixture of scaffold, sugar phosphodiester backbones bearing target analyte binding molecules, target anlytes, and permitting all of the binding reactions (both for construction of the complex and for binding of the target analyte) to take place simultaneously or nearly simultaneously.

In another embodiment, the complex is built in two reactions, the first of which attaches the scaffold to a solid support by any of a variety of means known in the art, and the second of which contains a mixture of sugar phosphodiester backbones bearing target analyte binding molecules and target anlytes, and permitting all of the binding reactions (both for construction of the complex and for binding of the target analyte) to take place simultaneously or nearly simultaneously.

### Manufacturing of Diagnostic Tests

The invention provides a method and means for the manufacture of diagnostic test or ligand capture strips, sheets or surfaces. The method or means includes a medium for deposition, a liquid deposition device for depositing, and a reagent to be deposited. The liquid deposition device includes any device capable of depositing small quantities of liquid, which can be directed to deposit the liquid in a regular or programmable pattern. In order for the test strips to be affordable (i.e. relatively low cost) and manufacturable at most locations, including remote and less civilized locations, quickly and without much operator intervention, the device should be inexpensive, relatively small in size, portable, programmable, and simple to operate. Exemplary preferred devices include printers, particularly inkjet printers, and particularly wherein the printer can be used with replaceable cartridges. A particularly preferred inkjet printer is the Hewlett-Packard deskjet printer. An additional preferred inkjet printer is a Lexmark printer.

A diagnostic test strip includes any regular or predetermined pattern of reagent(s) applied to a medium, including paper, nylon, plastic, filter or other surface. The regular or predetermined pattern may be lines, dots, bars, boxes, letters, symbols or images and can be placed in a linear, vertical, horizontal, circular or angled pattern.

Reagent(s) include a ligand, antigen, receptor, antibody, peptide, target sequence, active site, lectin, a component in a multicomponent complex, etc., in other words any component which can be bound to or by or otherwise stably interact with another component in a sample, solution or mixture.

The pattern may incorporate one or more than one reagent(s). Thus one reagent may be printed in a particular pattern or location and a second, third, etc. reagent may be printed in a different location or pattern. Instead of printing individual strips for each diagnostic or assay, for example, one strip can be printed in a series of lines running horizontally (e.g., bottom to top) or as vertical lines or locations next to one another (e.g. left to right). In this manner a test strip can assay for multiple components or diagnose for multiple diseases simultaneously. Each location or line indicates the presence or amount of a different component. Thus, a single test strip can cost-effectively and simultaneously assay, for example, for HIV, hepatitis B, hepatitis C, influenza, etc., as in a blood testing situation. One approach to such a multi-reagent printing is to utilize the different color vials (e.g. cyan, magenta, yellow) in a color inkjet printer. Each color vial can print a different reagent or can be used to print different combinations of reagents. Alternatively, the strip may be consecutively printed by reloading the print medium or paper and printing a different reagent on the strip as in overprinting. The inventors have successfully overprinted over a dozen times without problems.

Also, the printer may use a multi-component reagent, as in for instance a library of antigens, peptides, compounds or phage to print on a strip. The antibody or binder will bind to its target from the multi-component mix on the strip. The antibody or binder can then be released physically or chemically.

The medium includes paper, particularly paper which has a nylon, acrylic, plastic or other water-resistant or protective surface or coating. The paper includes inkjet paper, glossy paper, Whatman paper. Track etched membranes may also be used.

A conventional (e.g. first world) manufacturing and distribution model for rapid diagnostic test manufacture and development involves a centralized manufacturing facility where components are assembled. Assembled components are then distributed from the central location. The need for up-front acquisition of expensive manufacturing equipment to manufacture such assays can create a formidable barrier to assay deployment. To address this issue, we propose a rapid diagnostic assay-manufacturing model in which a liquid deposition device, an inkjet printer for example, is employed to “print” such assays with components either obtained from a quality controlled central source or locally manufactured. To address the issue of manufacturing equipment expense, we employed (as an example, although not limiting in the current invention) a low-end HP deskjet printer for deposition of the capture reagent on such assays. Advantages of the method include that no modifications to the printer are required and antibody printing involves simply replacing the ink in an HP27 (black ink cartridge) with the capture antibody solution.

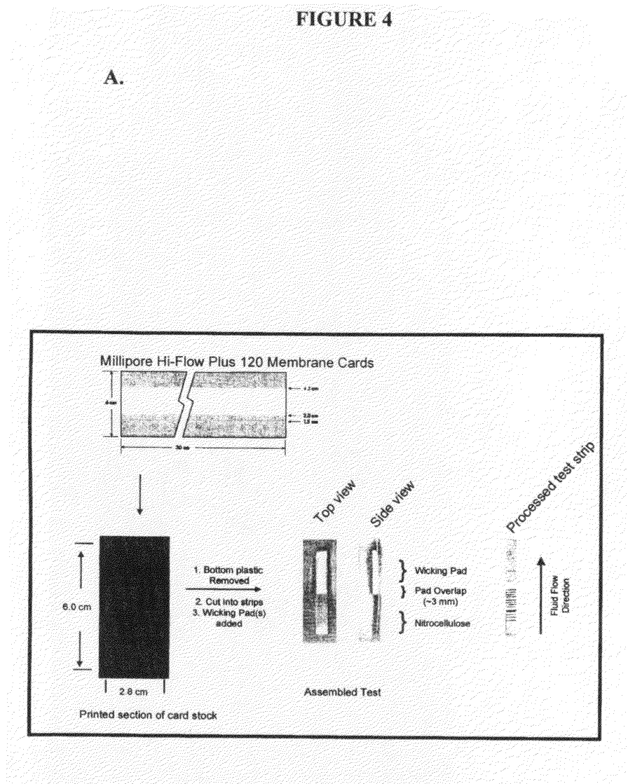

This invention provides for the use or modification of an existent printer, particularly an inkjet printer, and/or construction of a new printer which provides the user with a relatively simple and portable manufacturing approach to immunochromatographic diagnostic assay manufacture. Without limitation and as an example only, FIG. 4 illustrates a Hewlett-Packard deskjet printer and the minor modifications to the inkjet cartridge required to employ the printer in to manufacture an antibody based immunochromatographic diagnostic assay. Further steps in the manufacturing process are provided, as exemplary material and without limitation on the actual assembly process employed or materials therein, are given in FIG. 4.

The various aspects of the present invention allow for a method for distributed manufacture of diagnostic tests comprised of a test format amenable to local manufacture and execution, e.g., the methods of the present invention; an inkjet printer; printable test media; a mixture containing antibodies and/or antibody constructs amenable to inkjet printing, said mixture being in any of a variety of forms include frozen, liquid, or dried which would require rehydration prior to use; various other test components as anticipated by the methods of the present invention; and a pattern or program for printing, which may be encoded in a computer system attached to the printer (e.g., a figure in a drawing program) or may be encoded on a memory card for which an interface slot is provided on the printer, or by other encoding means known in the art. This method offers economic benefits by permitting distribution of the various components to the test manufacture site, even permitting such distribution from multiple, disparate sources. Further benefits accrue from the use of local (to the point of manufacture or point of use) personnel at prevailing, local wage factors, thereby offering significant cost reduction over a single point of manufacture.

The methods for distributed manufacture of diagnostic tests may include use of software that permits or requires license enforcement for licenses regarding the manufacture and use of a diagnostic test that includes license terms, which software may use communications facilities, e.g., the Internet, to communicate with a licensing authority to permit manufacture of the test or to control aspects of the test manufacture, e.g., the number of tests that may be printed.

Local manufacture can include, for example, manufacture of the assembly in proximity to the location at which the diagnostic test will be executed, e.g., at a doctor's office, at a clinic, at a local warehouse, etc. The more remote the location, the greater the advantage conferred by the present invention.

Advantages conferred by the present invention include, but are not limited to, economic advantages, e.g., local manufacture is often less expensive than centralized manufacture and distribution; shipping of components instead of completed assemblies permits choice of shipping method for each type of component, thereby further increasing the economic advantage; and, local assembly permits shipping of components in their most stable forms.

In one embodiment, the present invention is comprised of a system of aspects working cooperatively to effect the local manufacture and assembly of the diagnostic assay. The aspects are delineated below, and it is obvious to one skilled in the art that the order of presentation does not imply or suggest priority or prerequisite of one aspect over another unless explicitly indicated.

One aspect of the present invention employs a device for liquid deposition onto a medium, for instance but not limited to, an inkjet printer, which is used to apply capture reagents onto the medium in repeatable volumes over repeatable patterns, e.g., bands, spots, lines, or other such shapes and/or layouts as are required by the diagnostic assay. The deposition device may include a computer system to provide control over the deposition process, or the pattern or patterns may be defined on a memory device which is plugged into or is otherwise read by a printer or other deposition device, or, the printer or deposition device itself may have, internally defined, controlling patterns for deposition.

Another aspect of the present invention employs a medium which is useful for creating lateral flow diagnostic tests, for instance but not limited to nitrocellulose-coated acrylic, upon which the aforementioned liquid deposition device may deposit diagnostic reagents in patterns, e.g., bands, spots, lines, or other such shapes and layouts as are required by the diagnostic assay. For purposes of the present discussion, medium upon which has been deposited diagnostic reagents is called “printed medium”.

Another aspect of the present invention includes a reagent or reagents that will be deposited upon the aforementioned medium to effect a critical component of the diagnostic assay, e.g., the target capture reagent. These reagents may be liquid or solid, and may be packaged in a form, e.g., solid, which is particularly resilient in shipping, and which is then resuspended in liquid form prior to introduction into the aforementioned liquid deposition device. Alternatively, these reagents may be shipped at a higher concentration of active ingredient(s) than will be used in the actual assay, thereby reducing the volume and/or weight of material to be shipped.

Yet another aspect of the present invention is comprised of any of a number of different methods for shipping materials, reagents and/or equipment (“material”), including, but not limited to, trucking or automotive, train, and aircraft, including both private and commercial providers of such shipping methods, or combinations thereof.

In a preferred embodiment of the present invention, the various matter comprising the diagnostic test components are shipped to a local manufacture site, at which the components are assembled, e.g., resuspension of capture reagents; the component(s) to be deposited onto the printed medium is/are placed into the liquid deposition device; the liquid deposition device is employed to deposit the components onto the medium, thereby resulting in printed medium; the printed medium is assembled with other required components thereby resulting in a complete diagnostic assay.

In a preferred embodiment, the liquid deposition device is an inkjet printer.

In another embodiment, the liquid deposition device is a device specifically designed to perform the manufacturing task of the present invention.

In another embodiment, liquid deposition device is programmed to require an operator validation step, part of which may optionally include requiring communication with an intellectual property holder to enable licensed printing of one or more printed medium.

In another embodiment of the present invention, the liquid deposition device obtains, either with or without operator intervention, patterns for deposition and/or license information for validation and enforcement by means of any of a variety of communications devices known in the art; for example, the device may require entry of a validation code that has been obtained by any communication means, so that the device is enabled to perform the liquid deposition. Further, the device may obtain, by any communication means, patterns for deposition of the materials specific to the particular assay under manufacture.

In another embodiment, the communication means includes any of telephone, satellite phone, Internet, wireless network, wireless device, Bluetooth, or network.

In another embodiment, an operator of the liquid deposition device employs the Internet or other communication means to order or purchase a number of patterns and/or a number of printed media enablements to be programmed into the liquid deposition device, and the liquid deposition device prints only those patterns and numbers of printed media as have been programmatically enabled.

In another embodiment, a dedicated machine capable of printing a variety of diagnostic assays is employed in a local environment. Such machine may be preprogrammed with specifications for assays, driven by an internet delivered or other remote programming. The machine may, optionally, report back on the diagnostic assay quality for quality control purposes or deliver diagnostic results for epidemiological purposes.

As suggested earlier, the diagnostic method of the present invention comprises examining a cellular sample or medium by means of an assay including a binding-scaffold. Patients or individuals capable of benefiting from this method include those suffering from cancer, a pre-cancerous lesion, a viral infection, a bacterial infection or other like pathological derangement.

The present invention further contemplates therapeutic compositions useful in practicing the therapeutic methods of this invention. A subject therapeutic composition includes, in admixture, a pharmaceutically acceptable excipient (carrier) and one or more of a binding scaffold, or fragment thereof, as described herein as an active ingredient. In a preferred embodiment, the composition comprises an antigen or target capable of modulating the specific binding of the antibody within a target cell.

The preparation of therapeutic compositions which contain peptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, adjuvants, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the peptide, polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic scaffold, nucleic acid, polypeptide or antibody containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle. The compositions are administered in a manner compatible with the dosage formulation, and in a therapeutically effective amount. The quantity to be administered depends on the subject to be treated, capacity of the subject's immune system to utilize the active ingredient, and degree of inhibition or neutralization of binding capacity or activity desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and subsequent administration or booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

As used herein, “pg” means picogram, “ng” means nanogram, “ug” or “μg” mean microgram, “mg” means milligram, “ul” or “μl” mean microliter, “ml” means milliliter, “l” means liter.

The labels most commonly employed for in the assays and methods of the invention are radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. A particular detecting material is anti-rabbit or anti-mouse antibody prepared in goats or other animals and conjugated with fluorescein through an isothiocyanate. The scaffold or its binding partner(s) can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

In a further embodiment of this invention, commercial test kits suitable for use by a medical specialist may be prepared. In accordance with the testing techniques discussed above, one class of such kits will contain at least a labeled antibody or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence or capability of cells for predetermined binding activity, comprising:

(a) a test strip manufactured or formatted as described herein;

(b) a predetermined amount of at least one labeled immunochemically reactive component obtained by the direct or indirect attachment of the antibody or a specific binding partner thereto, to a detectable label;

(c) other reagents; and

(d) directions for use of said kit.

More specifically, the diagnostic test kit may comprise:

(a) a test strip manufactured or formatted as described herein;

(b) a known amount of the antibody as described above (or a binding partner) generally bound to a solid phase to form an immunosorbent, or in the alternative, bound to a suitable tag, or plural such end products, etc. (or their binding partners) one of each;

(c) if necessary, other reagents; and

(d) directions for use of said test kit.

In accordance with the above, an assay system for screening potential drugs effective to modulate the activity of the antibody or target may be prepared.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

A Multivalent Anti-CD4 Cell Avidity Construct Employing an Anti-CD45 Receptor Antibody

In this example, an oligonucleotide of sequence 5′-CTAGCTCTACTACGTGGCTG-3′ is conjugated to anti-CD45 (eBioscience; see protocol).

Exemplary Oligonucleotide: Conjugation Protocol

An analyte-specific reagent for binding human CD4 cells was prepared as described below. The reagent included an anti-CD45 portion and an oligonucleotide “tail”. Specifically, human anti-CD45 IgG (available from eBiosciences) in 5 mM EDTA was reduced with 2-mercaptoethylamine hydrochloride (MEA, Pierce, Rockford, IlL) in buffer A (100 mM sodium phosphate, 5 mM EDTA, pH 6.0) to cleave the disulfide bond between the F(ab) fragments and provide a free sulfhydryl group. When the reaction was complete (incubation was at 37° C. for 90 minutes), the mixture was diluted with sterile buffer B (20 mM sodium phosphate, 150 mM NaCl, 1 mM EDTA, pH 7.4) and purified on a Bio-Rad Econo-Pac 10DG column, eluting with Buffer B. Fractions were collected and assayed for protein with a BCA assay (BCA Protein Assay Reagent kit, Pierce), being careful to distinguish false positives due to the reducing reagent (MEA). The protein-containing fractions were pooled and the yield was calculated. An assay for determination of free sulfhydryl groups (Ellman's reagent) indicated that each antibody fragment had several sulfhydryl groups. 3′-terminal amine-modified (dT).35 was obtained from Oligos Inc., and treated with sulfo-succinimidyl-4-(N-maleimidomethyl)cyclohexane-1-carboxylate (Sulfo-SMCC, Pierce, 25 mole equiv.) in sterile PBS (20 mM sodium phosphate, 150 mM NaCl, pH 7.2), to derivatize the (dT).sub.35 amino group. The reaction was typically incubated for 60 minutes at room temperature or for 30 minutes at 37′ degree. C. The derivatized oligonucleotide was purified (on a Bio-Rad Econ-Pac column eluting with Buffer B). Fractions containing modified DNA were detected by measuring the UV absorbance at 260 nm. The derivatized DNA was then conjugated to the cleaved F(ab) fragments prepared from anti-human IgG (molar ratio of modified DNA to protein was 10:1) by incubation for at least 2 hours (or overnight) at 4.° C. The conjugate was purified with a Centricon 60 centrifuge filter (Amicon) to provide the analyte-specific reagent.

The conjugated anti-CD45 construct can be employed as: 1) a monovalent T cell binder when no scaffold is provided, 2) a divalent T cell binder if the sequence 5′-TTTTTTTTTTTTTTTTTTTTTTTTTCAGCCACGTAGTAGAGCTAGGACAT CAACTCCAGACCATACAGCCACGTAGTAGAGCTAGGACATCAACTCCAG ACCATA-3′ is employed as a scaffold for the anti-CD4 antibody: oligonucleotide construct, or, 3) a multivalent binder of a variety of valencies if the complex of 2) is hybridized to poly d(A) [e.g. the dT₂₅ section of these molecules will allow assembly onto poly-d(A)n, if desired]. However, the divalent construct can be used in the absence of poly-d(A)n to assess the degree of avidity that the complexes display.

### Demonstration of Successful Construction of a Simple Avidity Nucleic Acid Scaffold Using a Streptavidin:Alkaline Phosphatase Surrogate in Place of Oligonucleotide Linked Antibody

Twelve lines of alkaline phosphatase: streptavidin were printed at a concentration that gave a known baseline signal at fifteen minutes treatment with BCIP/NBT color generator (e.g. ˜2.3×10¹⁰ molecules per line for a total number of printed streptavidin: alkaline phosphatase (Peirce) of ˜2.7×10¹¹ total molecules in all lines each line constituting a printed volume of ˜170 nanoliters). Next, both strips were placed in 1 mL 0.5% casein for 10 min. to block non-specific sites on the membrane. Next, either 200 uL tris-buffered saline (control) or dT25 oligonucleotide (obtained from Integrated DNA Technologies, Inc) in 200 uL tris-buffered saline (amplification) providing a total of ˜3×10¹² molecules dT25 or (approximately a ten fold excess of dT25 to total printed streptavidin: alkaline phosphatase) was allowed to flow up the membranes. Next poly d(A) (Sigma-Aldrich) approximately 100 to 200 bases in length was allowed to flow up the membranes. The poly d(A) was at a concentration that was “copy number limited” in that the total number of printed streptavidin: alkaline phosphatase molecules contained in all twelve printed lines on each strip was approximate 40% greater than the total number of poly d(A) molecules allowed to flow up the membrane (i.e. total number of poly-d(A) molecules was ˜1.0×10¹¹ copies). Then preformed dT25:streptavidin alkaline phosphatase was allowed to flow across both control and test strips. The preformed complex was made at a ratio of 1.2 copies of streptavidin:alkaline phosphatase to dT25 so that only dT25 complexed alkaline phosphatase was available to bind to that poly d(A).

Amplification of approximately 4-fold was obtained indicating successful building of a tetravalent complex which depleted as successive printed bands were encountered.

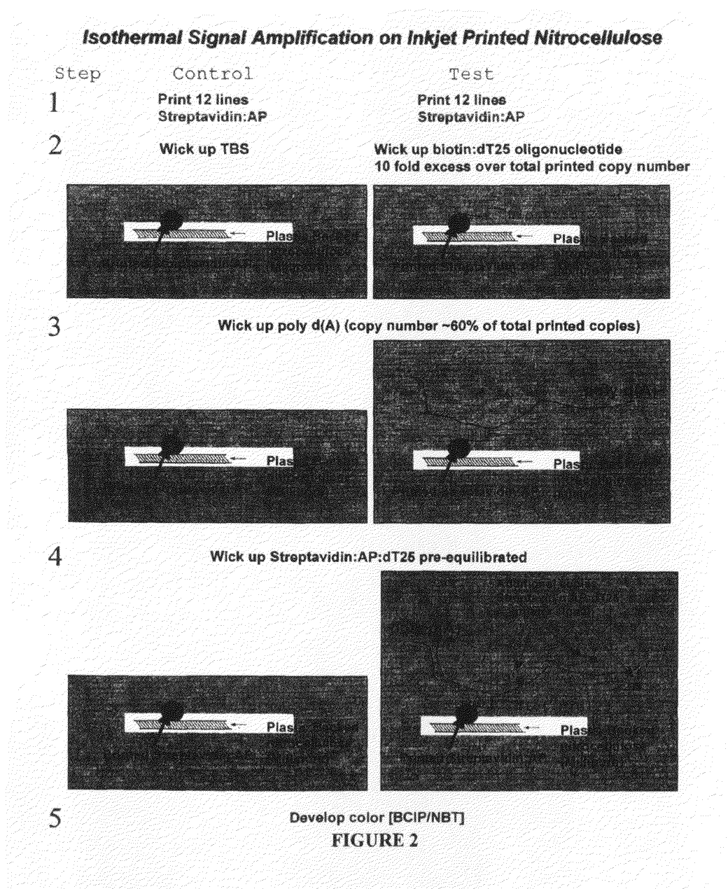

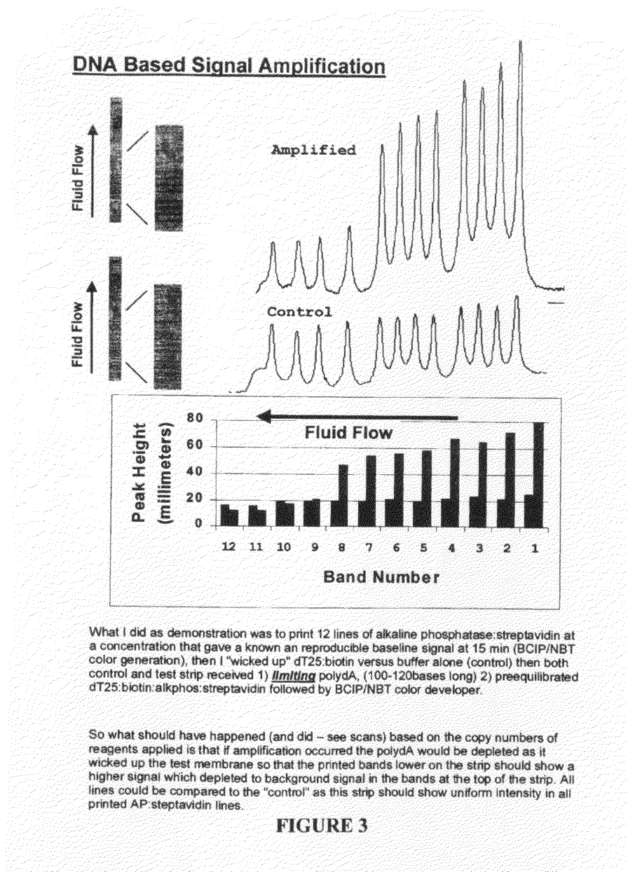

FIGS. 2 and 3 outline this example, using a streptavidin: alkaline phosphatase surrogate antibody marker to monitor DNA scaffold formation, and the results obtained. FIG. 2 depicts the isothermal signal amplification scheme on inkjet printed nitrocellulose. FIG. 3 depicts the results of amplified versus control using BCIP/NBT color generation to view the signals. One expects that if amplification occurred, the polydA would be depleted as it wicked up the test membrane so that the printed bands lower on the strip show a higher signal. The higher signal in the lower bands on the strip would be expected to (and do) show a higher signal which depletes to background level signal in the bands at the top of the strip. All lines should be compared to the “control” as this strip should show uniform intensity in all printed AP:streptavidin lines.

References Cited:
Hubble, J. “A model of multivalent ligand:receptor equilibria which explains the effect of multivalent binding inhibitors” (1999) Molecular Immunology 36 13-18
Hubble et al., 1995;
Hubble, 1997;
Daniak et al., 2006
Schweitzer et al., (2000) “Immunoassays with rolling circle amplification” PNAS 97: 10113-10119; supplementary materials
J. E. Gestwicki, C. W. Cairo, L. E. Strong, K. A. Oetjen and L. L. Kiessling (2002). Influencing Receptor—Ligand Binding Mechanisms with Multivalent Ligand Architecture, J. Am. Chem. Soc. 124, 14922-14933.
Gowers, D. M. and Fox, K. R. (1999) Towards mixed sequence recognition by triple helix formation. Nucleic Acids Res., 27, 1569-1577.

### Example 2

### Inkjet Printed Lateral Flow Assay

This example depicts the simple manufacture of rapid diagnostic assays, by printing a reagent onto a medium for deposition using a liquid deposition device, in this exemplary instance printing onto nitrocellulose test strips using an HP inkjet printer. Tests are printed onto nitrocellulose “card stock” using an Inkjet printer on an “as needed” basis (FIG. 4A). Printing involves opening an HP27 print cartridge, removing the black ink and foam followed by rinsing extensively with water. Then the “screen” over the printhead is removed carefully with tweezers. The print cartridge is then extensively rinsed again with water followed by printing distilled water continuously over an entire page to “purge” the printhead of any remaining ink residue. Then 200-250 microliters of antibody/protein solution is added (spiked with yellow food dye to monitor printing). Any pattern may be constructed in a graphics package (e.g. Microsoft Powerpoint) and printed.

The Assembly steps for an inkjet printed lateral flow assay may include the following (FIG. 4B): 1) Millipore lateral flow card stock is cut to desired size (i.e. depending on number of test strips desired), taped to 8.5×11 in. paper and antibody (or other protein) printed. The printed card stock is then cut into 3 mm “strips”. Optionally, a “wicking pad” is attached such that it overlaps the nitrocellulose by ˜2-3 mm.

### Example 3

### A Rapid and Quantitative CD4 Test

### Specific Aims

This example involves initial development and validation of a rapid, quantitative lateral flow (immuno-chromatographic) CD4+ T cell counting assay. Our approach to capture of CD4+ cells relies on construction of inexpensive “avidity” constructs capable of capturing all CD4+ cells as they flow across a nitrocellulose membrane. The avidity constructs are applied to the nitrocellulose membrane using ink-jet deposition and the focus of this initial study is to validate the avidity capture approach in the dipstick format. The results of this study will be used to construct an inexpensive dipstick-based CD4+ T cell counting assay that can be used under non-laboratory conditions to obtain clinically relevant assessments. The aims of this study include:

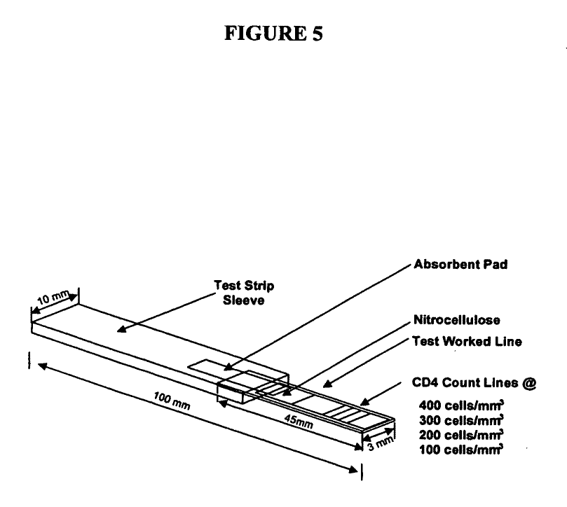

(1) Construct and quantify the effects on T cell binding of antibody:DNA avidity constructs with a variety of anti-CD2 receptor “valencies”.
(2) Empirically determine and minimize the steps needed for producing a prototype anti-CD4 dipstick lateral flow assay. The basic features of our dipstick design can be seen in FIG. 5.

### Background and Significance

The total HIV positive patient population worldwide is in excess of 40 million. The vast majority of individuals living with this disease are in resource poor environments where conventional CD4+ T cell enumeration is both too expensive to perform and technically challenging, due to a paucity of trained personnel. Treatment efforts currently underway, such as the World Health Organizations “3 by 5” Initiative, will be providing access to HAART (e.g. highly active anti-retroviral therapy) to millions of patients in these areas of the world over the next several years.

It is in such resource poor environments where CD4 counts are arguably the most important to perform. Current costs and assay complexities limit this. An accurate CD4 count can be employed: to facilitate AIDS surveillance; to monitor the rate of progression to AIDS, to define when therapy is required to prevent opportunistic infections, to place drug-naive patients into cohorts prior to therapy, and to monitor the effects of anti-retroviral therapy (c.f. Jani et al., 2001, 2002; www.affordCD4.com). It is currently recommended that a CD4 assay is performed on every HIV-infected individual every 3-6 months (MMWR; 1997; 46:1) and more frequently depending on circumstance. The assay described here is intended to answer this need, both from the standpoint of addressing the technical difficulties and the cost requirements.

Current CD4 counting assays are expensive, especially in resource poor settings and generally require some technological sophistication for assay execution. The gold standard for such testing is cell sorting. Currently available assays and their estimated costs are summarized in Table 1.

TABLE 1
CD4 Tests and Their Cost^{a}

| Test | Munufacturer | Equipment Required | ~Cost* |
|---|---|---|---|
| FACSCount | Becton | Flow cytometry instrument; | US$~40.00 |
| | Dickinson | automated | |
| Cytosphere | Beckman | microscope, | US$~15.00 |
| | Coulter | haemocytometer; manual | |
| Dynabeads | Dynal | mixer, magnet, microscope; | US$~16.00 |
| CD4/CD8 | | manual | |
| Capcellia | BioRad | plate reader, magnet, | US$~10.00 |
| | | multichannel pipette; | |
| | | manual | |
| Easy | Guava | micro cytometry | US$~7.50 |
| CD4/CD8 | Technologies | instrument, computer; semi- | |
| | | automated | |
| Partec | Partec | dedicated cytometer, | US$~5.00 |
| CyFlow | | computer; semi-automated | |

^{a}Adapted from Balikrishnan et al., 2005

Table 1 illustrates that even the “lower cost” tests represent a significant cost burden in resource poor environments. Even the lowest cost test (not accounting for labor) is of significant cost with respect to the estimated $181.00 per patient year expected expense for ART therapy once local drug manufacturing is available (Badri et al., 2006) if CD4 counts are to be useful for monitoring infected individuals. It is also significant that all of the tests described above require some type of instrumentation with attendant training and specialized environment associated with its use (for review see Balkrishnan et al., 2005; Constantine and Zink, 2005). Constantine et al., 2005 also list the following tests as available: Opti-CIM (CIMA, light microscopy, price not available), Zymmune (Zynaxis Corp, withdrawn from market), TRAxCD4 (T Cell DXs and Immunogenetics; withdrawn from market), CD4 Count Chip (SemiBio, no pricing available) and CD4 Biochip (Labnow, launch this year, pricing unavailable). Also, cited pricing varies from source to source although the $3-10 range is agreed upon for most manual tests.

A variety of approaches to reduce costs in existent assays have been reported (reviewed in Rodriguez et al., 2005). “PanLeucogating” (c.f. Glencross et. al., 2002) and use of “generic”, i.e. not proprietary, antibodies (c.f. Pattanapanyasat et al. 2005) have both been evaluated; however, the need for additional commercial reagents and the “center-based” deployment of cytometric devices is a difficult burden to overcome. A prototype microchip based methodology for CD4 counting in resource-limited environments has recently been described (Rodriguez et al., 2005), however, as has been pointed out by others (Bentwich, 2005) the final cost of the device and associated reagents is unknown at this time.

Some larger corporations have withdrawn CD4 count tests from the market (c.f. Zymmune and TRAxCD4). In the developed world, flow cytometry is the available gold standard and there is little impetus for changing this. In the underdeveloped world, this option is not only non-affordable but also requires a high degree of technical sophistication. CD4 counting assays that have been designed to fill this need, while certainly more affordable than flow cytometry, still require either equipment and/or technical sophistication to perform. From this perspective, the argument could be made that there is very little profit motive to develop and market such tests. First world requirements for approval of new diagnostic tests present an additional monetary barrier for corporations, which, for all practical purposes must show either a profit or the potential for it. Yet, if the pricing scheme for such a test is not as low as possible, the test will not be deployed where it is most needed.

One advantage of this approach is that it can be manufactured locally, if the assay is designed with the appropriate attributes, such a test will generate first world interest in the avidity-based lateral flow strategy.

### The Lateral Flow CD4 Test

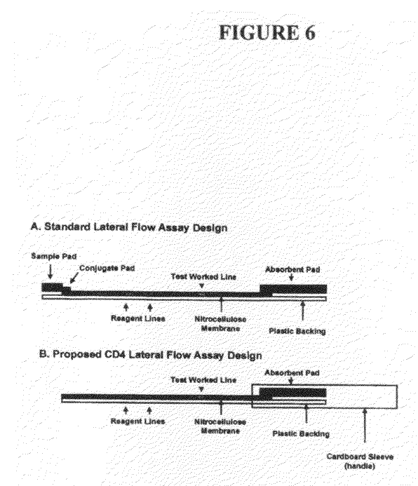

Lateral flow point of care assays have become commonplace in drug testing, pregnancy testing, etc. and have been shown to be remarkably robust to the variation they are exposed to as home test solutions (c.f. Zeytinoglu et al., 2006) if care is taken in assay design (Jacobs et al., 2001). Such assays, when sold in the first world, are generally one-step sample application (blood, urine, saliva, etc.) tests with the assay encased in plastic (reviewed in von Lode, 2005). A typical lateral flow assay design is shown in FIG. 6. Unfortunately, objective intensity assessment for the purpose of “counting” analyte captured using standard capture reagents (monoclonal antibody, streptavidin: biotin, etc.) can only be objectively accomplished with reader instrumentation to assess final staining intensity of the capture band, despite claims of unaided visual endpoints (reviewed thoroughly in von Lode, 2005). True counting with this approach would also be expected to require standard environmental requirements (temperature dependence of both equilibrium binding and color generation steps, respectively). The actual field assay we have designed can be described as a series of “steps”. The patient uses a glass capillary to perform a “finger-stick” which collects 0.10 mL of whole blood. This blood sample is then deposited into a vial containing 200 uL platelet wash buffer consisting of 148 mM NaCl, 5 mM glucose, 0.6 mM EDTA, and 20 mM Tris, pH 7.4 (Bessos and Murphy, 2002). The vial is lightly shaken to disperse the blood throughout the solution and the dipstick is inserted into the vial to allow the entire solution to “wick up” through the strip. After the entire blood sample has been depleted, the dipstick is moved to a second vial containing 0.30 mL of “blocking reagent”, containing casein, which serves also as a “wash” solution. After this solution has been depleted, the dipstick is moved to a third vial that contains 0.10 mL of a solution containing anti-CD4 antibody coupled to alkaline phosphatase (AP). The last step is a detection step using BCIP/NBT color generator contained in a fourth vial. Thus the entire “test kit” contains four plastic screw-cap vials, one glass capillary tube to perform the needle stick and one dipstick.

The precise design is made possible by the final avidity capture reagent quantitatively binding all CD4-expressing cells which flow across the avidity capture “lines”. The quantitative capture afforded by the avidity reagent allows the test to be interpreted as follows: if only the first line is visible, the original 0.10 mL blood sample contained fewer than 100 CD4 positive cells/uL, if the first two lines are visible the sample contained between 200 and 300 CD4 cells/uL, if the original blood sample contained 300 CD4 cells/uL but less than 400 cells/uL the sample will darken the first three lines and if all four lines are visible the sample contained greater than 400 CD4+ T cells/uL.

### Attributes of the CD4 Assay

In the ideal case, a CD4 assay suitable for resource poor environments would have several critical attributes we believe must be addressed and accounted for so that the final product represents the desired attributes. In this section, these attributes are described. Our overall exemplary design is based on the “Capcellia” strategy which employed an anti-CD2 monoclonal antibody to capture all T-cells and a secondary (anti-CD4/CD8) “staining” antibody (Carrière et al., 1999; Kannangai, 2001).

### Attribute 1. The Assay Must be Easy to Manufacture

The sheer volume of required tests is daunting. If we accept that 1 million people will be receiving ART in the developing world at the end of 2005 (WHO; 3by5 report; June, 2005) then ideally (at four tests per year) 4 million assays would be required. This number will multiply dramatically by 2010.

Approach: A conventional (e.g. first world) manufacturing and distribution model is not appropriate for this volume of tests, if they are to be made available in a timely fashion. It would take a minimum of several years to “scale up” to this level of test production. During this time, tests would not be available in the areas where they were most needed. Therefore, one parameter that must be considered is that the test must be capable of being manufactured locally on an “as needed” basis. The need for up-front acquisition of expensive manufacturing equipment to manufacture the assay would create a formidable barrier to test deployment. To address this, we propose a lateral flow assay (plastic-backed nitrocellulose strip) with ink jet deposition of the CD4+ T cell capture (avidity) reagents. We have already defined the “strip” size such that a total of 100 assays can be printed per Millipore Hi-Flow “card” of lo mil plastic backed nitrocellulose. To address the issue of equipment expense, we have employed a low-end HP deskjet printer (DeskJet Model 3945; US$ 39.90; Wal-Mart) for deposition of the capture reagent. No modifications to the printer are required and antibody printing involves simply replacing the ink in an HP27 (black ink cartridge) with the capture antibody solution (at appropriate concentration). For test design and printing, we employed Microsoft Powerpoint software. Printing was monitored by inclusion of trace quantities of yellow food dye.

Attribute 2. The Assay Must be Capable of being Used in a Variety of Physical Environments by Unskilled Personnel.

Approach: The attribute allowing for the performance of the test by an unskilled operator is addressed by employing a simple, four-step assay which requires only that the operator of the test move the test strip sequentially from vial to vial, and then interpret the results by visually assessing the number of stripes that appear when the test is complete (−30 min.). The issue of environment control in a classically-distributed test would lead immediately to long term stability studies with all components, especially when reagents are stored at ambient temperatures. However, the approach proposed here allows for the critical reagents to be maintained in a controlled environment up to and including a local distribution point, from which test kits can be prepared and assembled for short-term distribution and use on an “as needed” basis.

### Attribute 3. The Assay Must be Able to “Count” CD4 Cells/uL at Appropriate Levels Using a Colorimetric Approach to Avoid the Need for Machine Reading of Test Output.

Approach: The ability to count CD4+ T cells using antibody detection methodology is, of course dependent on the “signal generation” yield and signal to noise expectation (and equipment for data interpretation). For example, fluorescent signal generation is generally associated with lower backgrounds giving better detection of a given target molecule due to improved signal to noise ratio (versus a colorimetric approach). Here, we focus exclusively on colorimetric detection as we want the final test to be interpreted by eye, using untrained personnel. The most inexpensive and common reagent to employ in an ELISA reaction, which generates a colorimetric endpoint, is Alkaline Phosphatase (AP) using BCIP/NBT as substrate. Given this constraint, the question arises: Can the colorimetric approach be reasonably expected to produce a visually observable signal at the levels of CD4 cells relevant to the problem? The answer comes down to assessing both the number of AP molecules necessary to generate detectable signal (detection limit) and the number of CD4 receptors which an anti-CD4:AP conjugate would be expected to encounter at the requisite CD4 cell counts for the assay. Preliminary results spotting AP on the nitrocellulose substrate we are currently using generates a detection limit of ˜10⁹ copies of AP (signal generation after 15 minutes at room temperature—data not shown). Human CD4+ cells average 10⁵ copies of the receptor per cell (Lenkei and Andersson, 1995), and using these values we can determine whether a colorimetric approach is feasible. The CD4 “counting” levels we have defined are 100 cells/mm³, 200 cells/mm³, 300 cells/mm³, and 400 cells/mm³, although additional count lines could be introduced if desired. Assuming that 100 ul of whole blood serves as the sample, then at the 100 cells/mm³ level, ˜10⁹ CD4 copies will be available for binding, which is sufficient to produce a visible colorimetric signal even at the 100 cells/mm³, albeit uncomfortably close to the detection limit. If some additional signal is desired or required, we employ a non-proprietary robust non-enzymatic means to amplify the result up to several thousand-fold (Lane et al., 1997, 2001) to aid in routine visualization. Regardless of actual background encountered in field-based use of the assay, the ability to generate additional signal as needed is expected to be sufficient to overcome any potential signal generation issues.

### Attribute 4. The Readout Must be Visually Interpretable by Untrained Personnel.

Approach: The physical design of the assay, as a series of colored stripes on a test strip, provides an intuitive interpretation modality. Provided the operator of the test can:
1) count the presence of stripes in the measurement domain of the test strip (color interpretation is not necessary as BCIP/NBT produces a dark insoluble precipitate), and,
2) confirm the presence or absence of control signals in the control domain of the test strip, the proposed assay will provide clear results.
   The two domains of the test strip will be physically separated from one another. This attribute requires that virtually 100% of the of the avidity capture reagent “lines” capture a defined and reproducible number of T cells as they flow across the membrane.

### Attribute 5. The Assay Must be Substantially Free of Existent Intellectual Property Constraints.

There is little point in developing this assay if it cannot be used without the burden of first world licensing fees and associated cost structures due to employment of either patented processes or compositions of matter. An overriding principle in our current design of the CD4 assay is that as designed, it is composed of methods and compositions which avoid proprietary processes and compositions, i.e. the methods and compositions we have devised are already in the public domain. We reasoned that if we employed only technologies that we knew were either unencumbered or were in the public domain by both U.S. and international patent law, uncontrolled costs due to licensing could be avoided. For example, with respect to the use of immuno-chromatographic strips (nitrocellulose, etc.), a fair number of public domain patents (c.f. Gould et al., 1985; Tom et al., 1982; Deustch and Mead, 1978; Valkirs et al., 1986 and references therein) exist, which make it clear that the general process is free from intellectual property constraints. Similarly, ink jet deposition of biological materials (antibody, DNA, etc.) has also existed for a surprisingly long period of time and analysis of expired patents (c.f. Johnson, 1980; Sangiovanni and Michaud, 1982; and references therein) reveals that simple ink-jet deposition of biomolecules onto a substrate does not appear to be IP-constrained. Other required steps are also in the public domain. For example, oligonucleotides must be conjugated to antibodies to construct the avidity reagents and this chemistry has been known for decades (Smith, 1976; Batz et al., 1981). The decision to use colorimetric (BCIP/NBT) detection was also driven by consideration of cost, as many of the dyes in current assays are proprietary (for example the vast majority of Invitrogen Corporation, aka Molecular Probes, dyes are quite expensive and require a license for commercial use). As far as the avidity constructs are concerned, any of various oligonucleotide-based signal amplification schemes can be used herein. In one exemplary approach, linear polynucleotide is used from a signal amplification scheme (Lane et al., 1999, 2001, U.S. Pat. Nos. 5,902,724 and 6,245,513). In this scheme, hundreds of coupled dT₂₀:FITC₂ molecules were hybridized to polydA with detection via anti-FITC antibody coupled to alkaline phosphatase. This yielded a greater than 10³ fold amplification signal (Lane et al., 1999, 2001). These patents are directed to methods and kits using the amplification method (not compositions of the DNA structures) and these patents also utilize polyd(A) of greater in length than 3000 nucleotides.

### Quantitative Lateral Flow Assays

Point of care (POC) assays based on lateral flow principles, represent a cost-effective choice when compared to alternative tests (c.f. Branson, 2000 for review). Lateral flow tests have recently been approved for the diagnosis of HIV infection (reviewed in Constantine et al., 2005, Branson, 2004). In addition, the dipstick assay design is used for drug testing, pregnancy testing, blood typing, infectious agent detection, monitoring of cardiac enzyme levels, water testing and a variety of other more specialized applications. They are relatively easy to design (depending on application of course) and the raw materials from which such assays are constructed are available commercially from a variety of sources. Interestingly, despite their widespread use, the theoretical parameters regarding capture of analyte have not been rigorously defined (see Qian and Bau, 2004; Qian and Bau, 2003 for review) and consequently, reduction of such tests to practice and manufacture has largely been driven by empirical principles (cf. Weiss, 2001; Oraskar, 1999). Further, while detection of a particular analyte using, for example, an antibody capture reagent, is fairly straightforward, designing these assays to be quantitative, such that “counting”, as is required in a rapid CD4 assay, can be accomplished without the need for equipment (i.e. with a visually interpretable endpoint), has been difficult.

The Difficulty of Counting Using the Lateral Flow Approach without Instrumentation

To understand why it is difficult to visually “count” with such assays consider the issue(s) involved. A typical lateral flow device uses a capture reagent applied to a membrane such as nitrocellulose in order to “capture” an analyte as it “flows” over the capture reagent (see FIG. 2). Detection may be performed simultaneously or subsequently with a secondary antibody (e.g. “the detector”; colorimetric, fluorescent, etc.) analyte binder analogous to a standard ELISA. While one can standardize flow rates, capture reagent zone size and volumes, in the end, the capture reagent:analyte equilibrium affinity is finite and some analyte inevitably will “escape”. Essentially, as analyte becomes bound to the capture reagent, the effective concentrations of analyte and capture reagent are reduced to the point that the reaction is no longer thermodynamically favored. [Note that this applies to wash steps as well, where there is zero analyte concentration in the solution flowing over the membrane.] This “binding constant effect” can, of course, be demonstrated but is intuitively obvious as one would always prefer a capture reagent:analyte affinity which is as high as possible (for this reason the vast majority of such assays employ monoclonal antibodies for capture and detection of analyte). One can, of course, create a standard curve of signal intensity versus concentration (for any given test configuration) from which the number of analytes bound to the capture reagent could be estimated fairly well. This would necessitate some means (equipment) to accurately measure band intensity, and would make the test more difficult to control as environmental variables (temperature, time, etc.) would then have to be rigorously controlled. Both of these issues add undesirable attributes to the final test design (cost and technical sophistication required).

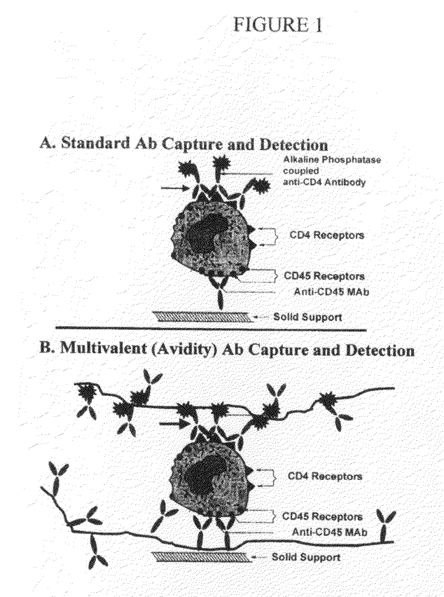

This attribute, the ability to actually count the cells flowing across the membrane without the need for some type of equipment to “read” the result, was/is the most daunting (from a technical perspective) that we wish(ed) to incorporate into this new CD4 assay. In effect, what we have designed is a DNA:antibody avidity capture approach which is able to quantitatively capture all T cells (using an anti-CD2 antibody) flowing across the membrane. Avidity is a term that describes the interaction between multivalent substances. Our version of an avidity capture strategy is shown in FIG. 1. Making the assumption that any CD4+ T cells caught by the capture reagent can be detected, what we are proposing is to increase the apparent affinity of the anti-CD2 antibody by employing it as a polyvalent construction. In effect, we are exploiting the polyvalency (multiple copies) of the CD2 receptor on the cell surface by allowing these receptors to bind to our polyvalent anti-CD2 constructs. This will increase the valency of the CD2 and anti-CD2 interaction which will lead to a “bonus” binding effect due to cooperativity of the association and dissociation of the observed binding reaction (versus monovalent binding to the receptor). To state this in an alternative fashion, the probability that all anti-CD2 antibody interactions will dissociate simultaneously becomes exceedingly small as the number of anti-CD2:CD2 interactions increases, if the anti-CD2 antibodies are linked together (c.f. Hubble, 1997, Minga et al., 2000). One antibody dissociating from a single receptor will not cause the complex to dissociate. In addition, the spatial localization of any dissociated antibody: antigen complex enhances the probability that any particular dissociated interaction will re-associate more quickly than when the reactants are free in solution. In effect, the dissociation reaction will be approaching zero at some level of anti-CD2 antibody “chaining”.

In general (i.e. as depicted in FIG. 3A), the interaction of anti-CD2 (the “capture reagent”) with

its ligand can be described by the standard free energy relationship for two interacting species, e.g.

ΔG=−RT ln Ka (1)

However, given that CD2 is a polyvalent molecule receptor on T cells, if we make the capture reagent polyvalent for the CD2 receptor by coupling anti-CD2 antibodies together using a linear polymer, we would have the requisite parameters for an avidity capture reagent where the free energy governing the reaction becomes:

ΔG_{avidity}=Σ₁^{|n-m|}f(ΔG) (2)

or, in terms of the equilibria involved

K_{avidity}=Π₁^{|n-m|}f(Ka) (3)
[where: n=number of anti-CD2 antibodies in avidity construct, m=number of CD2 receptors available for binding, ΔG=Gibbs free energy, R=universal gas constant, T=absolute temperature, and f is an adjustable parameter describing the apparent increase

In observed binding reaction per additional anti-CD2]

Of course, this effectively statistical description, while retaining the expected relationship from the interaction of two polyvalent species interacting, does not take into account the “geometry” of the binding elements (CD2 receptor and anti-CD2 antibody). The CD2 receptor could appear in dense clusters on the cell surface or be dispersed randomly or display some combination of these extremes across the surface. However, from a purely statistical description and assuming that there are no steric issues, we can expect that with as few as 10 anti-CD2:CD2 interactions from any given coupled anti-CD2 avidity construct would make it unlikely that the interaction could be displaced by monovalent anti-CD2 at any reasonable concentration (Hubble et al., 1995; Hubble, 1997; Daniak et al., 2006).

The constructs we initially have employed, albeit with a different antibody attached, have previously been utilized as “linear amplification reagents” for both antibody (ELISA) assays and to visualize DNA reactions on an ELISA plate without the need for PCR (or other enzymatic amplification steps, Lane et al., 1999; Lane et al., 2001). In this previous work, we demonstrated, using dT and polydA amplification constructs, that such constructs could be employed to amplify the sensitivity of analyte detection by an antibody up to several thousand fold. In brief, to effect this amplification the “detector antibody” is covalently linked to a oligo-dT₃₅ which is used to bind a long (several thousand bases) polydA molecule followed by attachment of signaling antibody:AP conjugate attached to dT₂₀ oligomers. The long polydA can accommodate hundreds of dT₂₀ oligomers (Lane et al., 1999, Lane et al., 2001).

Experiments with Inkjet Deposition of Antibody

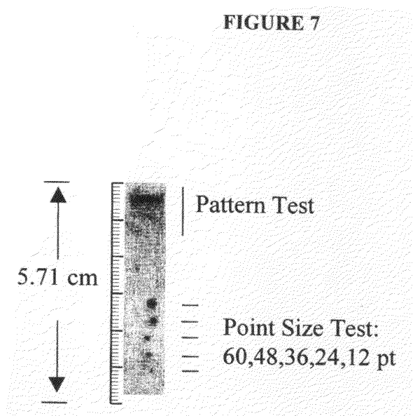

While the inkjet deposition of biomolecules is actually quite common, use of a standard office printer is less so. In considering the problem, there are two basic types of inkjet processes to choose from, either piezoelectric (Epson) or “bubblejet”/thermal (Canon; Hewlett Packard). HP print heads are quite easy to use (and replace as each cartridge itself contains a new printhead) and despite the thermal bubble ink ejection process, biomolecule activity appears to be retained (Thomas Boland, Clemson University—personal communication). The antibody printing was remarkably simple to reduce to practice using the Inkjet printer and the resolution was quite surprising (see FIG. 7), even on print paper or media (Azon). We have also utilized a variety of other substrates (nitrocellulose, genescreen, etc.), employing nitrocellulose substrates as being less likely to allow diffusion during post-printing manipulations. We adopted the “lateral flow” approach in which reagents were allowed to wick vertically up the membrane. The steps involved in printing and processing are summarized in FIG. 4B.

### Demonstration of Quantitation Approach for Assay Development

The ability to assess the degree to which multivalent constructs are assembled and perform during development of this assay was not addressed in the first submission of this proposal. In FIGS. 2 and 3 (above) we document our ability to create and quantify the suggested constructs (with streptavidin: alkaline phosphatase as a surrogate for oligonucleotide-linked antibodies). The methodology employed and described in FIGS. 2 and 3 was developed to serve as standard protocol for assessing both construct formation and eventually cell binding by the multivalent antibody constructs. In brief, each band in a given strip will develop a baseline signal due to printed streptavidin: AP^{b} and the difference between the control signal and observed signal can be used as a direct quantitative measure of “detection”. The experiment documented in FIGS. 2 and 3 also illustrates the “control” one can exert over these lateral flow assays. To be more precise, examination of the “amplified” scan reveals that only the first eight “lines” show signal amplification. This is because we deliberately made the polyd(A) limiting in the reaction (18×10⁹ copies of streptavidin: AP applied over twelve lines per strip but only 9×10⁹ copies of polyd(A) supplied) which caused the polyd(A) to be depleted as it “flowed” up the strip to the point where none was capable of reacting with the top four printed strptavidin:AP printed lines, which gave only “background” signal.

### DNA Molecules to be Employed in Multivalent Constructs

The key technical issue is not demonstration that DNA constructs with bound antibodies can function as avidity (i.e. multivalent) binding reagents. Based on the successes reported by others using different backbone chemistries, it seems unlikely that this will present a problem (c.f. Gestwicki et al., 2002; Mourez et al., 2001; Cairo et al., 2001; Sulzer and Perelson, 1996; Liang et al., 1997; Dam et al., 2000; Griffith et al., 2004; Kiessling et al., 2000). The key issue is whether such an effect can be manipulated to produce a reagent capable of “capturing” T cells in a quantitative manner as they flow across the membrane, or in other words, the efficiency with which the dissociation constant can be driven toward zero when actually binding T cells.

We have been able to obtain two commercial preparations of polyd(A) 1) average chain length 125-150 bases (Sigma, as used above) and 2) a preparation averaging 1000 bases (Fluka; Sigma, not yet tested).

The following milestones are undertaken:

1. Monovalent and multivalent avidity constructs of commercial monoclonal anti-CD2 will be prepared and compared in their ability to capture Jurkat T lymphoma cells (CD2+, CD4+, ATCC TIB-152).
2. Multivalent anti-CD2 constructs are compared to streptavidin: biotin binding efficacy using temperature variation.
3. Polyclonal antibodies (non-proprietary) against CD2 and CD4 are prepared.
4. Ability to count T cells (Jurkat) is documented.
5. Multivalent constructs of polyclonal anti-CD2 antibodies are compared against monoclonal constructs.

Milestone 1. In this milestone, we demonstrate that the suggested avidity capture constructs perform advantageously over monovalent target capture. Specifically, as a monovalent anti-CD2 construct, biotinylated anti-CD2 (Ancell Corp.) is employed attached to printed streptavidin:AP [as described above for the 5′ biotin; d(T)25]. CD4+Jurkat T lymphoma cells (CD2+, CD4+, ATCC TIB-152) [maintained in RPMI 1640 with 10% heat-inactivated fetal calf serum, penicillin (100 U/ml), streptomycin (100 U/ml), L-glutamine (2 mM), and 50 uM b-mercaptoethanol] are used as the target for capture and are detected using alkaline phosphatase conjugated anti-CD4 (Pierce). Multivalent anti-CD2 constructs are prepared by again printing strepatavidin:AP followed by attachment of 5′-biotin; d(T)25 (as in FIG. 2), followed by polyd(A) addition and finally d(T)25:antiCD2 conjugate (Ancell Corp.) for assembly of the multivalent complex. Both monovalent and multivalent complex assembly are monitored before exposure to the Jurkat cells by detecting the anti-CD2 antibody with anti-mouse IgG:alkline phosphatase (Sigma) using identical strips made in parallel. Signal intensities on the strips are used for quantitation purposes with attention paid to maintaining signal within the linear response range of the scanner employed (an HP flatbed scanner was used). Competition Assays: we also intend to perform competition assays to assure that the system can capture CD2+ cells in the presence of competing cells and these experiments are performed with both the monovalent and multivalent constructs as follows, and results compared to those above. Prior to use, the cells are washed in PBS twice, and then counted. Normal adult levels of white blood cells are 4,500-11,000 cells/ul blood, or 4.5−11.0×10⁶ cells/ml. CD4+Jurkat cells, 1×10⁵ (100 cells/mm³), 2×10⁵ (200 cells/mm³) or 4×10⁵ (400 cells/mm³) are mixed with CD2-BC-3 B cell lymphoma cells (CD2-, CD4-, ATCC CRL-2277), so that the final cell number is 4.5×10⁶/ml (c.f. Barrett, 2002). The strips are blocked for 20 min. in 0.5% casein. One hundred microliters of the cell suspension are allowed to flow up the “test” strip while 100 ul TBS alone or containing 4.5×10⁶ BC-3 cells (as a negative control) are allowed to flow up the “control” strips. Strips are processed as in FIGS. 2 and 3. Since lymphocytes account for approximately 25-45% of the total white blood cell count, their normal range is 1,000-4,800 lymphocytes/ul of blood, or 1.0−4.8×10⁶ cells/ml. Of the total lymphocytes, approximately 45-60% are T cells. In a second experiment, CD4+Jurkat cells, again at 100, 200 or 400/mm³, are mixed with CD4-TALL-104 T lymphoblast cells (CD2+, CD4-, ATCC CRL-1 1386), to a total of 6.0×10⁵ T cells. BC-3 cells are then be added, so that the final cell number is 4.5×10⁶ cells/ml. The cell mixture is then tested for reactivity with the strips as described above. This allows us to evaluate the effect of competing CD2+CD4-cells (which would include CD8+CD2+T cells and CD4-CD2+NK cells) on the specificity and efficiency of the assay.

Milestone 2. In demonstrating that the multivalent capture reagent will perform better than the monovalent capture approach, we also determine the degree to which the multivalent constructs are enhanced. This is accomplished by comparing the results obtained from the experiments in Milestone 1 with those when the same experiment is performed at two additional temperatures (4° C. and 37° C., for convenience). Our expectation is that a monovalent construct shows a larger degree of dependence on the temperature at which it is performed than a multivalent construct does (lowered dissociation rate). For comparison, we run a simple streptavidin:biotin interaction study (biotinylated mouse IgG printed:detection with streptavidin: AP) at the same three conditions.

Milestone 3. Rabbits are immunized with purified soluble CD2 or CD4 (R & D Systems), in Hunters Titermax adjuvant, as previously described (Price et al., 2002; Denny et al., 2006). Immunoglobulin is isolated from serum by Protein G affinity chromatography, and concentrated. The specificity and titer of the anti-CD4 and anti-CD2 Ig will be determined by flow cytometry on Jurkat cells, using an anti-rabbit:phycoerythrin conjugated secondary antibody.

Milestone 4. Determining the absolute sensitivities of assays of this type (either Ab detection assays or nucleic acid based assays) require titration of all reagents against one another systematically and this milestone will be accomplished by such a strategy.

Milestone 5. The above avidity constructs are made with commercially available monoclonal antibodies. We will demonstrate the efficacy of antibodies we prepare employing the same approach as in Milestone 1, that is, conjugate the polyclonal mixture with d(T)25.

### Literature Cited

Badri M, Maartens G, Mandalia S, Bekker L G, Penrod J R, Platt R W, Wood R, Beck E J (2006) PLoS Med 3(1): e4
Balakrishnan P, Solomon S, Kumarasamy N Mayer K H (2005) Indian J Med Res 121 345-355. Barrett, J (2002) in Gale Encyclopedia of Medicine, December, 2002 by the Gale Group
Batz, H-G, Horn, J; Stellner, K; Maier, J; Nelboeck-Hochstetter, M; Weimann, G (1981) Hydroxy-succinimide ester compounds. U.S. Pat. No. 4,248,786.
Bessos, H, Murphy, W G (2002) “Competitive binding assay immunoassay for platelet antigens in whole blood.” U.S. Pat. No. 6,479,246.
Branson B M (2004) Aids Clin Care 16 39.
Branson B M. (2000) Journal of International Association of Physicians in AIDS Care; February:28-30.
Cairo C W, Gestwicki J E, Kanai M, Kiessling L L (2002) J AM Chem Soc 124 1615-1619.
Carrière D, Jean Pierre Vendrell, Claude Fontaine, Aline Jansen, Jacques Reynes, Isabelle Pagès, Catherine Holzmann, Michel Laprade, and Bernard Pau (1999). Clin Chem 45: 92-97.
Constantine N T, Kabat W, Zhao R Y (2005) Cell Research 15 870-876.
Constantine, N T and Zink, H (2005) Indian J. Med Res 121 519-538.
Dam T K, Roy R, Das S K, Oscarson S, Brewer C F. (2000) J Biol Chem 275 14223-14230.
Daniak M B, Kumar A, Galaev I Y Mattiasson B. (2006) Proc. Natl. Acad. Sci. USA 103 849-854.
Denny M F, Chandaroy P, Killen P D, Caricchio R, Lewis E E, Richardson B C, Lee K D, Gavalchin J, Kaplan M J. J. Immunol. 2006 Feb. 15; 176(4):2095-104.
Deutsch, Marshall E.; Mead, Louis W. (1985) Test device. U.S. Pat. No. 4,094,647
Gestwicki J E, Cairo C W, Strong L E, Oetjen K A, Kiessling L L (2002) J Am Chem Soc 124 14922-14933.
Glencross D, Scott L E, Jani I V, Barnett D, Janossy G. (2002) Cytometry 50 69-77.
Griffith B R, Allen B L, Rapraeger A C, Kiesslin L L (2004) J Am Chem Soc 126 1608-1609.
Hubble J, Eisanthal R, Welsh W J D. (1995) Biochem J. 311 917-919.
Hubble J. (1997) Immunol Today 18:305-6.
Hubble J. (1997) Immunology Today 305 27-28.
Jacobs E, K. A. Hinson, J. Tolnai and E. Simson (2001) Clin. Chim. Acta 307 49-59.
Jani, I., Janossy, G., Brown D W G and Mandy F. (2002). Lancet Infectious Diseases 2: 35-43
Jani, I., Janossy, G., Iqbal, A., Mhalu, F. S., Lyamuya, E. F., Biberfeld, G., Glencross D. K., Scott L. E., Reilly, J. J., Granger, V. & Barnett, D. (2001) Journal of Immunological methods 257: 145-154.
Johnson, L C. (1980) Method of making printed reagent test devices. U.S. Pat. No. 4,216,245.
Kannangai, R., Ramalingam, S., Jesudason, M. V., Vijayakumar, T. S., Abraham, O. C., Zachariah, A., Sridharan, G. (2001). Clin. Diagn. Lab. Immunol. 20 1286-1288.
Khandjian E. W., Biotechnology 5: 165-167, 1987
Kiessling L L, Strong, L E, Gestwicki J E (2000) Ann Rep Med Chem 35 321-330.
Lane, M J.; Benight, A S.; Faldasz, B D. (1999) Signal amplification method. U.S. Pat. No. 5,902,724.
Lane, M J.; Benight, A S.; Faldasz, B D. (2001) Signal amplification method. U.S. Pat. No. 6,245,513 B1.
Lenkei, R. & Andersson, B. (1995) J. Immunol. Methods 183, 267-277.
Liang R, Loebach J, Horan N, Ge M, Thompson C, Yan L, Kahne D. (1997) Biochemistry 94 10554-10559.
Minga F, Robert Eisenthala, Whisha W J D, Hubble J (2000) Enzyme and Microbial Technology 26 216-221.
Mourez M, Kane R S, Mogridge J, Metallo S, Deschatelets P, Sellman S E, Whitesides G M, Collier R J. (2001) Nature Biotech 19 958-961.
Oroskar, A A (1998) IVD Technol Jan 11. [Available online: www.devicelink.com/ivdt/98/01.html.]
Pattanapanyasat K, Thakar M R. (2005) Indian J Med Res 121 539-549
Pattanapanyasat, K, Hla Shain, Egarit Noulsri, Surada Lerdwana, Charin Thepthai, Varipin Prasertsilpa, Sirirat Likanonsakul, Prakit Yothipitak, Somboon Nookhai, Achara Eksaengsri (2005) Cytometry Part B: Clinical Cytometry 65B 29-36.
Price, K. D., Knupp, C. J., Tatum, A. H., Stoll, M., Jiang, F., Gavalchin, J. (2002). J. Autoimmunity 19(3): 87-101.
Sangiovanni, J J.; Michaud, R J. (1982) Ballistically controlled nonpolar droplet dispensing method and apparatus. U.S. Pat. No. 4,341,310.
Smith, (1976) Compound, dithiobis-(succinimidyl propionate). U.S. Pat. No. 3,940,420.
Sulzer S and Perelson A S. (1997) Math. Biosci 135 147-185.
Tom, H K.; Rowley, G L. (1982) Concentrating zone method in heterogeneous immunoassays. U.S. Pat. No. 4,366,241.
Valkirs G E, Owen C E, Levinson P A (1986) Method and Apparatus for Immunoassays. U.S. Pat. No. 4,632,901.
von Lode P (2005) Clin Biochem 38 591-606.
Weiss, A. (1999) IVD Technol November/December 42. [Available online: www.devicelink.com/ivdt/archive/99/11/009.html.]
World Health Organization (2005) 3 by 5 progress report: Available www.who.int/3by5/.
Zeytinoglu A, Turban A, Altuglu I, Bilgic A, Abdoel T H, Smits H L. (2006) Clin Chem Lab Med. 44 180-184.

### Example 4

### A Rapid Diagnostic Map Antibody Assay for Field Use

### Specific Aims

This example provides the development of a low-cost, easy quantitative lateral flow (immuno-chromatographic) Map antibody assay suitable for field use. Our approach to detection of Map antibodies relies on the construction of inexpensive “avidity” constructs capable of capturing all Map antibodies as they flow across a nitrocellulose membrane. The avidity constructs are applied to the nitrocellulose membrane using ink-jet deposition and the focus of this study is to validate the avidity capture approach in a dipstick format. The results are used to construct a quantitative inexpensive dipstick-based Map antibody assay that can be used under non-laboratory conditions. The specific aims are:

1) Construct and quantify the effects of Map antigen:DNA avidity constructs on Map antibody binding with a variety of recombinant Map antigen “valencies” and detectors in a dipstick design format.
2) Evaluate the performance characteristics of the Map antibody lateral flow dipstick assay and its specificity and sensitivity using sera from known Map-positive and negative cattle.

### Background and Significance

Mycobacteruim avium subsp. paratuberculosis (Map) causes a wasting disease, Johne's disease, that results in granulomatous lesions of the lymph nodes of the small intestine in ruminants [1-3]. According to the Johne's Information Center, it is estimated that 7.8% of the beef herds and 22% of the dairy herds in the U.S. are infected with Map. Animals apparently are infected when young but, while shedding the organism via feces, these animals may not show clinical symptoms for several years [4]. Because the organism resides in macrophages in the intestinal mucosa and associated lymph nodes [5, 6], infected animals may have reduced feed efficiency without obvious clinical signs of disease. At the present time, there is no treatment or vaccination program in the United States that effectively prevents Johnes disease [7]. Current control methods are based upon minimizing exposure to feces from infected animals in dairy herds, requiring early identification and culling of infected animals [8].

Currently, there are several different commercially available tests for the detection of Map infection. Some rely on detecting Mycobacterium paratuberculosis. Isolation of Map is the definitive test for the diagnosis of Johnes disease; however, culture techniques require 6 to 12 weeks to obtain a result, and performance is variable, due to lack of standardized culture conditions and the difficulty is growing the organism [9-11]. Nucleic acid based techniques like PCR are available, but these require specialized equipment, are expensive and also are less sensitive particularly in low shedding animals [12]. Other assays detect the production of Map-specific antibody in serum. Still another method to detect infection is by determining cellular immune responses to Map by skin testing [13]. Although some of the tests are simple enough to be able to be done in a veterinary clinic, in general they require sophisticated laboratory equipment and skilled laboratory technicians to perform them.

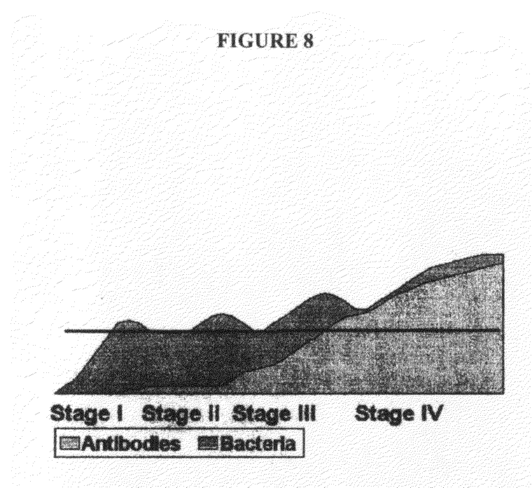

Detection of serum antibody to M. paratuberculosis is good evidence that an animal is infected [9, 10, 14-17]. The agar-gel immunodiffusion test for antibodies (AGID) is highly specific; however, it has a lower sensitivity in cattle and is most often used to confirm clinical diagnosis: i.e. to verify a diagnosis on animals with clinical signs of disease that looks like Johne's disease (diarrhea and weight loss). The Map ELISA can test large numbers of samples quickly, and is relatively low-cost. Another advantage is that Map antibody titers can be quantified by ELISA and the level of antibody may be useful in predicting the stage of infection. Furthermore, ELISA is more sensitive than AGID in cattle, and is nearly as sensitive as fecal culture at detecting infected animals. However, a disadvantage of the current Map antibody ELISA is that they require skilled laboratory technicians and specialized laboratory equipment. Furthermore, the current tests are unable to detect the lower levels of anti-Map antibodies that are produced in the early stages of disease [1, 10, 14, 16] (FIG. 8).

In general, most of the available tests for Johne's disease have a high specificity, and a low rate of false-positive results [9, 10, 17]. However, assay sensitivity (percentage of infected animals that test positive) varies among tests, but due to the biology of this slowly progressing chronic disease is often less than 50%. Tests have maximum sensitivity when used for animals with diarrhea and/or weight loss, the clinical signs of infection. However, in the very early stages of M. paratuberculosis infection, before animals start shedding the bacterium in feces or begin an immune response to the infection, there are no clinical signs of disease. Unfortunately, diagnostic tests that can detect disease in these animals are not available.

For a herd that is infected, the objective of an effective management program to control Map infection is to make the diagnosis early, in order to cull infected animals. A herd that is verified to not be infected will profit in terms of health status and profit loss, as well as in sale of dairy replacement cattle. However, while testing of a herd is recommended, the current tests are not amenable to this. First, all of the tests required trained personnel, and often the turnaround time required to obtain test results may be quite long. The cost of the test may also be prohibitive. Veterinary diagnostics for food animals are more strongly affected by end user economics than diagnostics for human diseases: there are no third party payers, and profit margins in animal agriculture are small [18]. With the losses in the US due to Johne's Disease at over $220 million/year [19, 20], there clearly is a need for a rapid, sensitive and easy-to-use assay to detect Map infection at the least cost.

### Preliminary Results

In this example, a low-cost, easy, and sensitive quantitative lateral flow (immuno-chromatographic) Map antibody assay is developed and validated for field use by dairy and cattle farmers. Our approach to the capture of Map antibodies relies on the construction of inexpensive “avidity” constructs of antigen capable of binding all Map antibodies as they flow across a nitrocellulose membrane. The actual constructs for this study have previously been employed as “linear amplification reagents” for both antibody (ELISA) assays and to visualize DNA reactions on an ELISA plate without the need for PCR (or other enzymatic amplification steps [21, 22]. In previous work, it was demonstrated, using the amplification constructs of dT and polydA, that such constructs could be employed to amplify the sensitivity of analyte detection by an antibody up to several thousand fold. In brief, to effect this amplification the “detector antibody” is covalently linked to a oligo-dT₃₅ which is used to bind a long (several thousand bases) polydA molecule followed by attachment of signaling antibody:AP conjugate attached to dT₂₀ oligomers. The long polydA can accommodate hundreds of dT₂₀ oligomers [21, 22].

Feasibility Experiments with InkJet Deposition of Antibody

The lateral flow assay is developed using plastic-backed nitrocellulose strips in order to deposit the avidity capture agent on the dipstick and chose to use inkjet deposition. While the inkjet deposition of biomolecules has been accomplished previously, we have utilized a standard office printer. In considering the problem, there are two basic types of inkjet processes to choose from, either piezoelectric (Epson) or “bubblejet”/thermal (Canon; Hewlett Packard). HP print heads were quite easy to use (and replace as each cartridge itself contains a new printhead) and despite the thermal bubble ink ejection process, biomolecule activity appears to be retained (Thomas Boland, Clemson University—personal communication). We are currently using an HP deskjet printer (DeskJet Model 3945) to “print” the capture reagent and no modifications to the printer are required. Antibody printing involves simply replacing the ink in an HP28 (black ink cartridge) with the antigen solution (at appropriate concentration). For test design and printing we employed Microsoft Powerpoint software. Printing was monitored by inclusion of trace quantities of yellow food dye. The antibody printing was remarkably simple to reduce to practice using the Inkjet printer and the resolution was quite surprising, even on printing paper (FIG. 7). We next utilized a variety of other substrates for the experiments (nitrocellulose, genescreen, etc.), finally settling on nitrocellulose substrates as minimizing the diffusion of reagents during post-printing manipulations. We have defined the “strip” size such that a total of 100 assays can be printed per 8½×11 sheet of nitrocellulose.

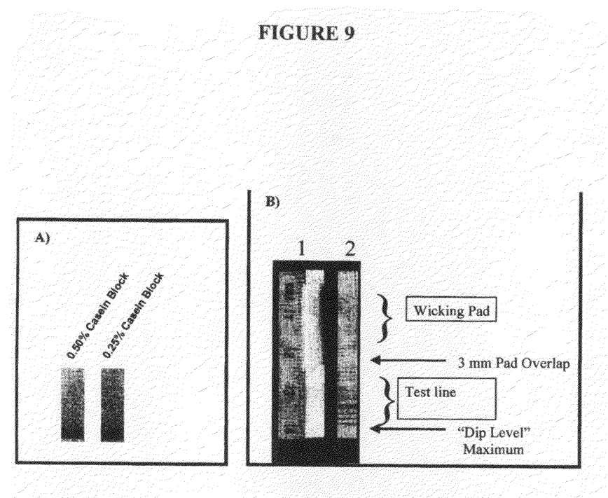

We next compare direct printing of Ab:AP conjugate (as in FIG. 7) with indirect detection (i.e. print a “target” antibody and then detect with an anti-antibody (i.e. print goat IgG and detect with anti-goat IgG: alkaline phosphatase conjugate and BCIP/NBT AP substrate). At this step we adopted the “lateral flow” approach in which reagents were allowed to wick vertically up the membrane. Initial attempts to detect in this fashion had background although the print pattern could be detected visually. Non-specific binding was reduced by the addition of 0.5% casein in a rinse step prior to adding detector conjugate. A comparison of results using 0.25% and 0.5% casein is shown in FIG. 9. It is worth noting that with this treatment, the resolution of the pattern was maintained with good signal to noise whether or not antibody: alkaline phosphatase conjugate is directly printed to the nitrocellulose or is allowed to flow up the membrane to detect a printed antigen.

Our experience with the initial nitrocellulose printing and detection reactions indicated that a plastic-backed nitrocellulose would be more convenient to work with. After a variety of plastic backed membranes were tested, Millipore Hi-Flow Plus 180 membrane cards (60 mm×301 mm) were used. This material has a ten mil plastic backing making the strips rigid. The cards were cut into an appropriate strip size post printing of antigen. An actual strip both before flow detection steps (left) and post detection steps (right-with the wicking pad removed) is shown in FIG. 3. In this experiment biotinylated goat IgG was printed to the nitrocellulose and detection was with streptavidin:AP conjugate (BCIP/NBT). The pattern is one we are employing to quantitatively examine capture efficiency given the Kd of 10^{13.5} for this equilibrium.

### Research Methods

Specific aim 1—Construct and quantify the effects of MAP antigen:DNA avidity constructs on MAP antibody binding with a variety of recombinant MAP antigen “valencies” and detectors in a dipstick design format.

We have developed a DNA:Map protein avidity capture approach which is able to quantitatively capture anti-Map antibody flowing across the membrane. Making the assumption that any Map antibody caught by the antigen can be detected, we increase the apparent binding of Map protein to anti-Map antibody by employing it as a polyvalent construction. Based on our previous work (see FIG. 1), we expect that this approach will improve the sensitivity of ant-Map antibody detection compared to current assays.

### Development of the Proposed Lateral Flow Map Antibody Test

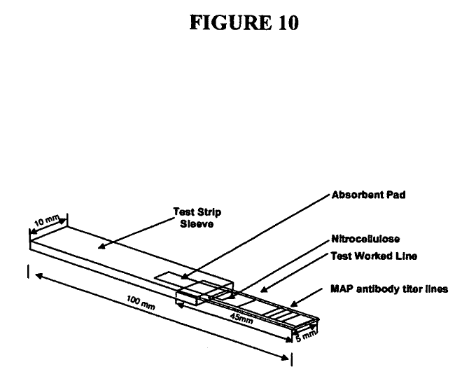

Lateral flow point of care assays have become commonplace in drug testing, pregnancy testing, etc. and have been shown to be remarkably robust to the variation they are exposed to as home test solutions [23]. Unfortunately, objective intensity assessment for the purpose of “counting” analyte captured using standard capture reagents (monoclonal antibody, streptavidin: biotin, etc.) currently available, can only be accomplished with reader instrumentation to assess final staining intensity of the capture band. True counting with this approach would also be expected to require standard environmental requirements (temperature dependence of both equilibrium binding and color generation steps, respectively), as well as experienced personnel to administer and interpret the test. The actual format of the field assay we envision would need to be optimized, but one example is shown in FIG. 10. A collection device like the BD microtainer capillary blood collection device (for whole blood or serum collection) would be used to collect 0.10 mL of whole blood from the ear of the animal to be tested. The sample is deposited into a vial containing, for example, 100-200 uL wash buffer consisting of 148 mM NaCl, 5 mM glucose, 0.6 mM EDTA, and 20 mM Tris, pH 7.4 [24]. Optimization both for the amount of buffer to add and its components may be required. The vial is lightly shaken to disperse the blood throughout the solution and the dipstick inserted into the vial to allow the entire solution to “wick up” through the capture lines. Successful wicking is indicated by the test line, which can be visualized by the RBCs, or as in our preliminary work, by the addition of a dye to the test sample. Another method, as described by Lou et al.[25], involves printing an indicator dye such as quinaldine red at the test line. The dye is colorless at pH<1.4 but turns red at pH>3.4. Thus a red color will appear once the sample crosses the test line. After the entire blood sample has been depleted, the dipstick is moved to a second vial containing 0.30 mL of “blocking reagent”, containing casein, which serves also as a “wash” solution. After this solution has been depleted, the dipstick is moved to a third vial that contains 0.10 mL of a solution containing anti-bovine Ig conjugated to alkaline phosphatase, at an appropriate concentration. If an additional amplification signal is desired, we employ a proprietary robust non-enzymatic means to amplify the result up to several thousand-fold [21, 22] to aid in routine visualization. Regardless of the actual background encountered in field-based use of the assay, the ability to generate additional signal as needed is expected to be sufficient to overcome any potential signal generation issues. The last step is a detection step using BCIP/NBT color generator contained in a fourth vial.

Many lateral flow assays are amenable to testing either whole blood or serum samples [26, 27]. The precise design is made possible by the assumption that the final avidity capture reagent quantitatively binds anti-Map antibodies which flow across the avidity capture “lines”. The results are easy to interpret. By determining how many lines are positive, ie reacting with substrate, the titer of Map antibody in the serum can be determined. Based on other lateral flow assay results, the assay should be completed within 15-20 minutes.

### Demonstration of Multivalent Capture Efficiency

A typical lateral flow device uses a capture reagent applied to a membrane such as nitrocellulose in order to “capture” an analyte as it “flows” over the capture reagent. Detection may be performed simultaneously or subsequently with a secondary antibody (e.g. “the detector”; colorimetric, fluorescent, etc.) analyte binder analogous to a standard ELISA. While one can standardize flow rates, capture reagent zone size and volumes, in the end the capture reagent:analyte equilibrium affinity is finite and some analyte inevitably will “escape”. Essentially, as analyte becomes bound to the capture reagent the effective concentrations of analyte and capture reagent are reduced to the point that the reaction is no longer thermodynamically favored. [Note that this applies to wash steps as well, where there is zero analyte concentration in the solution flowing over the membrane.] This “binding constant effect” can, of course, be demonstrated but is intuitively obvious as one would always prefer a capture reagent:analyte affinity which is as high as possible (for this reason the vast majority of such assays employ monoclonal antibodies for capture and detection of analyte). One can, of course, create a standard curve of signal intensity versus concentration (for any given test configuration) from which the number of analytes bound to the capture reagent could be estimated fairly well. This would necessitate some means (equipment) to accurately measure band intensity, and would make the test more difficult to control as environmental variables (temperature, time, etc.) would then have to be rigorously controlled. Both of these issues add undesirable attributes to the final test design (cost and technical sophistication required).

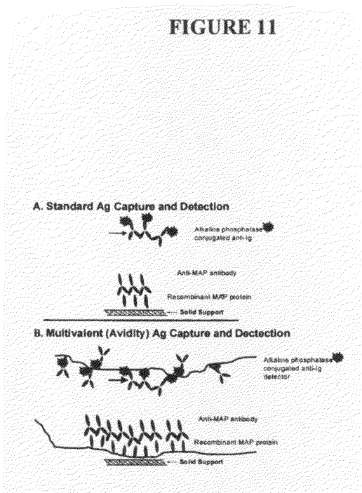

Our version of an avidity capture strategy is shown in FIG. 11. Making the assumption that any anti-Map antibody caught by the capture reagent can be detected, we increase the apparent affinity of the Map antigen by employing it as a polyvalent construction. In effect, polyvalent anti-Map antibodies are binding polyvalent Map antigen. This increases the valency of the Map/anti-Map interaction which leads to a “bonus” binding effect due to cooperativity of the association and dissociation of the observed binding reaction. To state this in an alternative fashion, the probability that all MAP/anti-MAP interactions will dissociate simultaneously becomes exceedingly small as the number of these interactions increases [28, 29]. In effect, the dissociation reaction will be approaching zero at some level of MAP “chaining”.

Based on the successes reported by others in attaining the same net effect using different backbone chemistries, we expect that that DNA constructs with bound proteins and antibodies can function as avidity (i.e. multivalent) binding reagents [30-37]. We have designed several oligonucleotides to test this in the following manner: We conjugate the oligonucleotide of sequence 5′-CTAGCTCTACTACGTGGCTG-3′ to one or more recombinant Map proteins. Several specific antigens of one or more Map proteins that elicit strong anti-Map responses during infection have been reported. These include the 85 A, B, and C complex, 35-kDAa (p35) and superoxide dismutase (SOD). Infected cows were found to produce detectable levels of anti-Map antibodies reactive with each of these recombinant proteins. Furthermore, the levels of antibody reactive with each of the recombinant antigens was increased according to shedding levels, and antibody to at least one of them, 35 kDa, was able to distinguish between healthy noninfected cows and cows shedding Map organism at both low and high levels (P<0.01). Thus, these proteins should be effective targets for antibody in our assay system. Recombinant plasmids for these proteins are obtained from Yung Fu Chang, Cornell University, and purified recombinant proteins for each of these antigens prepared as previously described [38, 39]. The purified Map proteins are used to prepare an analyte-specific reagent for detection of anti-Map using a method we have previously used to prepare anti-human IgG with a poly(dT) “tail”, as follows. Specifically, purified recombinant Map proteins in 5 mM EDTA are reduced with 2-mercaptoethylamine hydrochloride (MEA, Pierce, Rockford, Ill.) in buffer A (100 mM sodium phosphate, 5 mM EDTA, pH 6.0) to provide a free sulfhydryl groups. When the reaction is complete (incubation was at 37° C. for 90 minutes), the mixture is diluted with sterile buffer B (20 mM sodium phosphate, 150 mM NaCl, 1 mM EDTA, pH 7.4) and purified on a Bio-Rad Econo-Pac 10DG column, eluting with Buffer B. Fractions are collected and assayed for protein with a BCA assay (BCA Protein Assay Reagent kit, Pierce), being careful to distinguish false positives due to the reducing reagent (MEA). The protein-containing fractions are pooled and the yield is calculated. An assay for determination of free sulfhydryl groups (Ellman's reagent) monitors antibody fragment sulfhydryl groups. 3′-terminal amine-modified (dT).35 is obtained from Oligos Inc., and treated with sulfo-succinimidyl-4-(N-maleimidomethyl)cyclohexane-1-carboxylate (Sulfo-SMCC, Pierce, 25 mole equiv.) in sterile PBS (20 mM sodium phosphate, 150 mM NaCl, pH 7.2), to derivatize the (dT).sub.35 amino group. The reaction is typically incubated for 60 minutes at room temperature or for 30 minutes at 37′ degree. C. The derivatized (dT).sub.35 is purified (on a Bio-Rad Econ-Pac column eluting with Buffer B). Fractions containing modified DNA are detected by measuring the UV absorbance at 260 nm. The derivatized DNA is then conjugated to the cleaved F(ab) fragments prepared from anti-human IgG (molar ratio of modified DNA to protein was 10:1) by incubation for at least 2 hours (or overnight) at 4.° C.). The conjugate is purified using a Centricon 60 centrifuge filter (Amicon) to provide the analyte-specific reagent. [Note: Other protein conjugates have been derivatized with a (dT) 35 tail according to this procedure with only minor changes.]

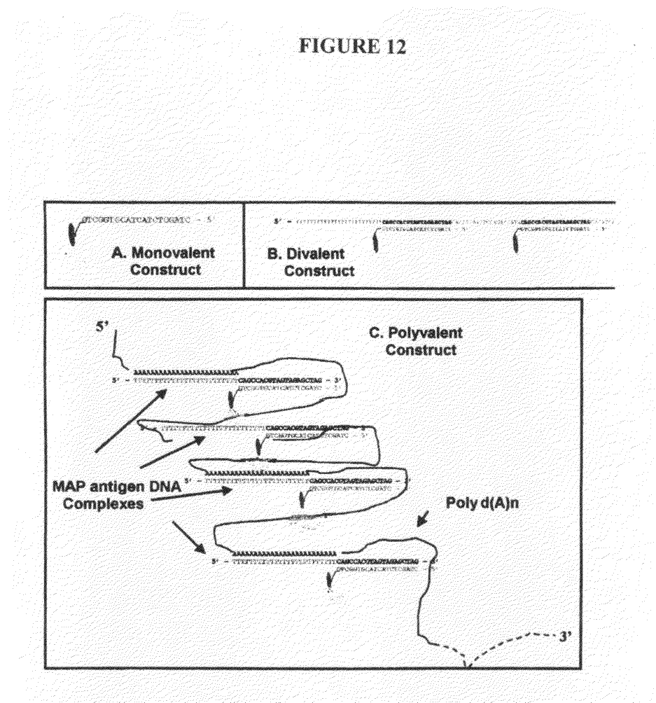

This conjugated Map antigen construct is employed as a monovalent anti-Map antibody binder (i.e. control) (FIG. 12). We have also obtained the two different complimentary oligonucleotides, both of which are 5′ tailed with dT₂₅. The dT₂₅ section of these molecules allows assembly onto polyd(A)n, if desired. However, the divalent construct can be used in the absence of polyd(A)n to assess the degree of avidity that the complexes display. All of the constructs are compared against biotinylated goat IgG binding to streptavidin: alkaline phosphatase as a control.

The polyd(A) is inkjet printed onto the nitrocelulose strips followed by UV (254 nm) irradiation of the printed polyd(A) membrane to affix the polyd(A) [40].

Next, the strip is blocked with casein as above to block non-specific binding of the Map antigen in the Map antigen: oligonucleotide construct solution. The Map antigen:oligonucleotide construct will then be assembled by allowing the solution to flow across the membrane.

Assay conditions including sample dilution, adsorption and buffer composition are optimized using positive control sera from high and low shedding cattle as well as matched negative control cattle obtained from the JDIP Diagnostic Core (Beth Harris, personal communication). The inclusion of an sample adsorption step may improve specificity, but could result in lowered sensitivity [41]. Maximizing test sensitivity is currently the biggest challenge for Johne's disease tests due to the biology of this infection. The goal is to improve the level of detection compared to the currently available assays, in order to detect antibody at the lower levels found in the early stages of infection.

Specific aim 2—Evaluate the performance characteristics of the MAP antibody lateral flow dipstick assay and its specificity and sensitivity using sera from known MAP-positive and negative cattle.

Repository samples available from the MIP Diagnostic Core are used to evalulate the performance of the Map lateral flow assay. Other well-characterized samples are provided by Yung Fu Chang, Cornell University [38, 39]. A total of 120 animals characterized as healthy controls (negative for Mycobacteruim avium subsp. paratuberculosis by fecal culture and IS900 PCR testing (n=40) and positive animals divided into low (1 to 30 CFU/g feces) (n=39), medium (31 to 300 CFU/g feces) (n=19) and high shedders (>300 CFU/g feces) (n=22), are available.

Determination of the Sensitivity and Specificity of the Assay by Testing Sera from Known Map-Positive and Negative Cattle.

The specificity of the Map lateral flow assay is determined by measuring the percentage of time a test result is negative for NON-infected animals (how well the test correctly identifies uninfected animals). Available blood tests for Johne's disease have a high specificity: 97% to 99% and culture-based tests are considered 100% specific (i.e., no false-positive tests). In general terms, this means that 97-99% of the time when a blood test is positive the diagnosis of Johne's disease is correct. A positive fecal culture correctly diagnoses Johne's disease 100% of the time.

The sensitivity of the assay, or the measure of the percentage of time that the lateral flow assay result is positive for infected cattle (how well the test correctly identifies infected animals) is also determined. Subtracting test sensitivity from 100% will give the percentage of infected cattle missed by the test (false-negative result).

### Establishment of Assay Dynamic Range

The dynamic range of the assay is determined by using the assay strips to assay a set of sera containing calibrated or known titers of anti-Map antibody [25]. Once the numbers of successive capture bars that should be stained at that concentration are identified then each anti-Map/anti-Ig-AP bound bar can be defined by a range of anti-Map antibody concentrations. In the case when all 4 bars are fully developed on the assay strip, the sample can be repeated after two-fold dilution in order to calculate the correct titer range.

### Testing of Assay Accuracy and Reproducibility

Twenty-five clinical samples with known anti-Map antibody titers previously determined by ELISA, and fecal load, obtained from the JDIP Diagnostic Core, are assayed in the Map antibody lateral flow assay. By comparing the number of capture bars developed on each strip with the calibrated tittered serum results above, the concentration of anti-Map antibody can be semi-quantitatively determined [25].

Reproducibility is determined by running the same sample more than once, as well as having different individuals interpret the results of the same set of assay straights. Agreement between data of multiple sets of data on the same sample and sets of data on the same sample obtained by different individuals is determined by linear regression.

### Stability of the Assay Strip

Strips prepared from the same printing are tested weekly with the same samples in order to determine the stability of the printed capture agent [25]. Stability and assay performance under conditions of different temperature and humidity is also evaluated.

### Literature Cited

1. Gay, J., Clinical description and epidemiology of Johne's disease in cattle. 2000, New York State Cattle Health Assurance Program.
2. Stabel, J. R, Transitions in immune responses to Mycobacterium paratuberculosis. Vet Microbiol, 2000. 77(3-4): p. 465-73.
3. Stehman, S. M., Paratuberculosis in small ruminants, deer, and South American camelids. Vet Clin North Am Food Anim Pract, 1996. 12(2): p. 441-55.
4. Kurade, N. P., et al., Sequential development of histologic lesions and their relationship with bacterial isolation, fecal shedding, and immune responses during progressive stages of experimental infection of lambs with Mycobacterium avium subsp. paratuberculosis. Vet Pathol, 2004. 41(4): p. 378-87.
5. Reddacliff, L. A., S. J. McClure, and R. J. Whittington, Immunoperoxidase studies of cell mediated immune effector cell populations in early Mycobacterium avium subsp. paratuberculosis infection in sheep. Vet Immunol Immunopathol, 2004. 97(3-4): p. 149-62.
6. Sigurethardottir, O. G., M. Valheim, and C. M. Press, Establishment of Mycobacterium avium subsp. paratuberculosis infection in the intestine of ruminants. Adv Drug Deliv Rev, 2004. 56(6): p. 819-34.
7. Harris, N. B. and R. G. Barletta, Mycobacterium avium subsp. paratuberculosis in Veterinary Medicine. Clin Microbiol Rev, 2001. 14(3): p. 489-512.
8. Collins, M. T., Clinical approach to control of bovine paratuberculosis. J Am Vet Med Assoc, 1994. 204(2): p. 208-10.
9. Kalis, C. H., et al., Evaluation of two absorbed enzyme-linked immunosorbent assays and a complement fixation test as replacements for fecal culture in the detection of cows shedding Mycobacterium avium subspecies paratuberculosis. J Vet Diagn Invest, 2002. 14(3): p. 219-24.
10. Reichel, M. P., et al., Comparison of serological tests and faecal culture for the detection of Mycobacterium avium subsp. paratuberculosis infection in cattle and analysis of the antigens involved. Vet Microbiol, 1999. 66(2): p. 135-50.
11. Whitlock, R. H. and C. Buergelt, Preclinical and clinical manifestations of paratuberculosis (including pathology). Vet Clin North Am Food Anim Pract, 1996. 12(2): p. 345-56.
12. Cousins, D. V., et al., Mycobacteria distenct from Mycobacterium avium subsp. paratuberculosis isolated from the faeces of ruminants possess IS900-like sequences detectable IS900 polymerase chain reaction: implications for diagnosis. Mol Cell Probes, 1999. 13(6): p. 431-42.
13. Robbe-Austerman, S., et al., Sensitivity and specificity of the agar-gel-immunodiffusion test, ELISA and the skin test for detection of paratuberculosis in United States Midwest sheep populations. Vet Res, 2006. 37(4): p. 553-64.
14. Jark, U., et al., Development of an ELISA technique for serodiagnosis of bovine paratuberculosis. Vet Microbiol, 1997. 57(2-3): p. 189-98.
15. Mutharia, L. M., W. Moreno, and M. Raymond, Analysis of culture filtrate and cell wall-associated antigens of Mycobacterium paratuberculosis with monoclonal antibodies. Infect Immun, 1997. 65(2): p. 387-94.
16. Rajukumar, K., et al., An enzyme-linked immunosorbent assay using immonoaffinity-purified antigen in the diagnosis of caprine paratuberculosis and its comparison with conventional ELISAs. Vet Res Commun, 2001. 25(7): p. 539-53.
17. Whitlock, R. H., et al., ELISA and fecal culture for paratuberculosis (Johne's disease): sensitivity and specificity of each method. Vet Microbiol, 2000. 77(3-4): p. 387-98.
18. Collins, M. T., et al., Evaluation of five antibody detection tests for diagnosis of bovine paratuberculosis. Clin Diagn Lab Immunol, 2005. 12(6): p. 685-92.
19. Losinger, W. C., Economic impact of reduced milk production associated with Johne's disease on dairy operations in the USA. J Dairy Res, 2005. 72(4): p. 425-32.
20. Ott, S. L., S. J. Wells, and B. A. Wagner, Herd-level economic losses associated with Johne's disease on US dairy operations. Prey Vet Med, 1999. 40(3-4): p. 179-92.
21. Lane, M., A. Benight, and B. Faldasz, Signal amplification method. U.S. Pat. No. 5,902,724, 1999.
22. Lane, M., A. Benight, and B. Faldasz, Signal amplification method. U.S. Pat. No. 6,245,513 B1, 2001.
23. Zeytinoglu, A., et al., Comparison of Brucella immunoglobulin M and G flow assays with serum agglutination and 2-mercaptoethanol tests in the diagnosis of brucellosis. Clin Chem Lab Med, 2006. 44(2): p. 180-4.
24. Bessos, H. and W. Murphy, Competitive binding immunoassay for platelet antigens in whole blood. U.S. Pat. No. 6,479,246, 2002.
25. Lou, S. C., et al., One-step competitive immunochromatographic assay for semiquantitative determination of lipoprotein (a) in plasma. Clin Chem, 1993. 39(4): p. 619-24.
26. Brooks, D. E., et al., RAMP(TM): A Rapid, Quantitative Whole Blood Immunochromatographic Platform for Point-of-Care Testing. Clin Chem, 1999. 45(9): p. 1676-1678.
27. Chan, C. P., et al., Development of a quantitative lateral-flow assay for rapid detection of fatty acid-binding protein. J Immunol Methods, 2003. 279(1-2): p. 91-100.
28. Hubble, J., Dissociation of multivalent antibody-antigen interactions. Immunol Today, 1997. 18(6): p. 305-6.
29. Minga, F., et al., Enzyme and Microbial Technology, 2000. 26: p. 216-221.
30. Gestwicki, J. E., et al., Influencing receptor-ligand binding mechanisms with multivalent ligand architecture. J Am Chem Soc, 2002. 124(50): p. 14922-33.
31. Mourez, M., et al., Designing a polyvalent inhibitor of anthrax toxin. Nat Biotechnol, 2001. 19(10): p. 958-61.
32. Cairo, C. W., et al., Control of multivalent interactions by binding epitope density. J Am Chem Soc, 2002. 124(8): p. 1615-9.
33. Sulzer, B. and A. S. Perelson, Immunons revisited: binding of multivalent antigens to B cells. Mol Immunol, 1997. 34(1): p. 63-74.
34. Liang, R., et al., Polyvalent binding to carbohydrates immobilized on an insoluble resin. Proc Natl Acad Sci USA, 1997. 94(20): p. 10554-9.
35. Dam, T. K., et al., Binding of multivalent carbohydrates to concanavalin A and Dioclea grandiflora lectin. Thermodynamic analysis of the “multivalency effect”. J Biol Chem, 2000. 275(19): p. 14223-30.
36. Griffith, B. R., et al., A polymer scaffold for protein oligomerization. J Am Chem Soc, 2004. 126(6): p. 1608-9.
37. Kiessling, L. L., J. E. Gestwicki, and L. E. Strong, Synthetic multivalent ligands in the exploration of cell-surface interactions. Curr Opin Chem Biol, 2000. 4(6): p. 696-703.
38. Shin, S. J., et al., Comparative antibody response of five recombinant antigens in related to bacterial shedding levels and development of serological diagnosis based on 35 kDa antigen for Mycobacterium avium subsp. paratuberculosis. J Vet Sci, 2004. 5(2): p. 111-7.
39. Dheenadhayalan, V., et al., Cloning and characterization of the genes coding for antigen 85A, 85B and 85C of Mycobacterium avium subsp. paratuberculosis. DNA Seq, 2002. 13(5): p. 287-94.
40. Khandjian, E. W., Biotechnology, 1987. 5: p. 165-167.
41. McKenna, S. L., et al., Comparison of two enzyme-linked immunosorbent assays for diagnosis of Mycobacterium avium subsp. paratuberculosis. J Vet Diagn Invest, 2005. 17(5): p. 463-6.

### Example 5

### Cellular Capture Assay

To demonstrate the application of the multivalent binding scheme to cell capture, assessment, isolation and analysis, the following was completed. CD4+Jurkat T lymphoma cells (CD2+, CD4+, ATCC TIB-152) [maintained in RPMI 1640 with 10% heat-inactivated fetal calf serum, penicillin (100 U/ml), streptomycin (100 U/ml), L-glutamine (2 mM), and 50 uM b-mercaptoethanol] were used as the target cells to demonstrate cell capture. Cells for the experiment were at ˜2×10⁶/mL initially and kept in media. A 100 ulL aliquot was supplemented with 20 ug biotinylated anti-CD4 mAB until 10 minutes before use when 10 uL of 0.5M EDTA (pH=8.0) was added to 90 uL of the cell suspension. The suspension was then pippetted onto a hydrophobic plastic surface to create a “bead” of cell suspension containing ˜2×10⁵ total cells.

Test “strips” were prepared as follows: 1) One third of a Millipore 065 nitrocellulose membrane card was taped to paper and 2) antibody lines “printed” by introducing anti-CD2 mAB into a type 27 HP print cartridge and using a pre-generated powerpoint file; 3) a ˜5 mm “strip” was cut from the printed membrane card and pretreated in 0.5% Casein “blocking” solution for 30 min. after which a “wick” was added to one end and 100 uL 1×TBS rinse was allowed to flow vertically across the membrane into the wick.

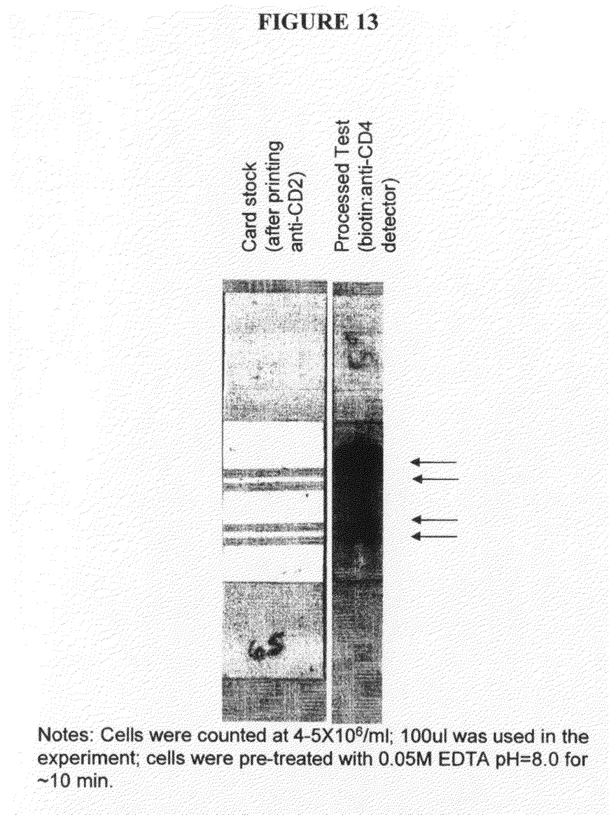

The strip was then immediately placed horizontally adjacent to the cell suspension “bead”. Physical contact with the cell suspension bead caused the cells to “wick” across the nitrocellulose (˜10 seconds). Immediately following the cell solution traversing the membrane the strip was placed into a well containing 100 uL TBS wash which was wicked vertically up the membrane. Next, 100 uL of streptavidin d(T)35 conjugate at a concentration of 0.05 pMoles/ul in TBS was added wicked across the membrane followed by a 100 uL TBS wash. Next, 100 uL of poly d(A) solution (Sigma) at a concentration 0.43 ng/uL was wicked up the membrane to convert the bound anti-CD4 to a polyvalent configuration followed again by a 100 uL TBS wash step. Signal was generated by allowing 100 uL of FITC d(T)20 conjugate to wick up the membrane and again a 100 uL TBS wash step. This was followed by allowing 100 uL of an anti-FITC: alkaline phosphatase conjugate at a concentration of 0.0670 pmoles/uL and BCIP for signal generation. The results are depicted in FIG. 13, the first strip is control after printing anti-CD2 and the second strip is the result showing positive cell bands captured at the printed anti-CD2 locations and visualized via anti-CD4.

This invention may be embodied in other forms or carried out in other ways without departing from the spirit or essential characteristics thereof. The present disclosure is therefore to be considered as in all aspects illustrative and not restrictive, the scope of the invention being indicated by the disclosure and description, including any appended Claims, and all changes which come within the meaning and range of equivalency are intended to be embraced therein.

Various references are cited throughout this Specification, each of which is incorporated herein by reference in its entirety.

What is claimed is:

1. A system for the capture of at least one analyte of interest in a sample, said system comprising:
(A) a substrate or solid support which is a wickable medium suitable for the reception and transport of said sample;
(B) a scaffold or polymer having a repeating unit, which scaffold or polymer is bound covalently or non covalently to the substrate or support of (A);
(C) a first capture reagent capable of binding directly or indirectly with analyte in the sample, which first reagent is affixed to or interspersed with the scaffold or polymer of (B);
(D) optionally a second capture reagent or binder, capable of binding (i) to both said first capture reagent and to an analyte in the sample or (ii) to a second analyte in the sample, which second reagent is affixed to or interspersed with the scaffold of (B) or which binds covalently or non covalently to the first capture reagent of (C);
(E) an indicator means which indicates that the sample has been transported along the substrate or support and confirms that the reagent(s) are operable.

2. The system of claim 1 further comprising a detector for quantifiable detection of analyte in the sample.

3. The system of claim 1 or 2 wherein
(A) the substrate or solid support is selected from glass, nylon, paper, nitrocellulose, and plastic;
(B) the scaffold or polymer is selected from nucleic acid, peptide, carbohydrate, and protein; and
(C) the first capture reagent is selected from antibody, antigen, peptide, nucleic acid, protein, ligand, carbohydrate, metal, fat, oil, and organic compound.

4. The system of claim 3 wherein the second capture reagent or binder is selected from antibody, antigen, peptide, nucleic acid, protein, ligand, carbohydrate, metal, fat, oil, and organic compound.

5. The system of claim 3 wherein the indicator means is a predetermined amount of analyte.

6. The system of claim 2 wherein the detector is selected from a label, radioactive element, enzyme, and dye.

7. The system of claim 2 wherein the detector is covalently attached to the first or the second capture reagent.

8. The system of claim 2 wherein the detector comprises an antibody, antigen, ligand, peptide, protein, nucleic acid or carbohydrate which binds or otherwise interacts with the analyte.

9. The system of claim 3 wherein one or more antibody serves as a first capture reagent and/or a second capture reagent or binder.

10. The system of claim 9 wherein the antibody is attached to the scaffold or polymer by means selected from noncovalent hybridization via sugar phosphodiester backbone hairpin structures and covalent attachment via chemical means.

11. The system of claim 3 wherein the scaffold or polymer is nucleic acid.

12. The system of claim 11 wherein the nucleic acid polymer or scaffold is a defined or repeating nucleic acid sequence.

13. The system of claim 11 wherein the first capture reagent comprises nucleic acid complementary to the nucleic acid sequence of the scaffold or polymer

14. A test kit for quantitation of one or more antibody or antigen in a sample comprising:
(A) a substrate or solid support which is a wickable medium suitable for the reception and transport of said sample and which is selected from glass, nylon, paper, nitrocellulose, and plastic;
(B) a scaffold or polymer having a repeating unit, which scaffold or polymer is bound covalently or non covalently to the substrate or support of (A) and which is selected from nucleic acid, peptide, carbohydrate, and protein;
(C) a first capture reagent capable of binding directly or indirectly with the antibody or antigen in the sample, which first reagent is affixed to or interspersed with the scaffold or polymer of (B) and which is selected from antibody, antigen, peptide, nucleic acid, protein, ligand, carbohydrate, and organic compound;
(D) optionally a second capture reagent or binder, capable of binding (i) to both said first capture reagent and to an antibody or antigen in the sample or (ii) to a second antibody or antigen in the sample, which second reagent is affixed to or interspersed with the scaffold of (B) or which binds covalently or non covalently to the first capture reagent of (C);
(E) an indicator means which indicates that the sample has been transported along the substrate or support and confirms that the reagents are operable, wherein the indicator is a predetermined amount of analyte; and
(F) a detector for quantifiable detection of antibody or antigen in the sample which detector is selected from a label, radioactive element, enzyme, or dye.

15. The test kit of claim 14 wherein the scaffold or polymer is nucleic acid and the first capture reagent comprises complementary nucleic acid, optionally attached to an antibody.

16. A method for the manufacture of an analyte capture strip to be used for capture of at least one analyte in a sample, which strip comprises
(A) a substrate or solid support which is a wickable medium suitable for the reception and transport of said sample, wherein the substrate is a printable medium;
(B) a scaffold or polymer having a repeating unit, which scaffold or polymer is bound covalently or non covalently to the substrate or support of (A);
(C) a first capture reagent capable of binding directly or indirectly with analyte in the sample, which first reagent is affixed to or interspersed with the scaffold or polymer of (B);
(G) optionally a second capture reagent or binder, capable of binding (i) to both said first capture reagent and to an analyte in the sample or (ii) to a second analyte in the sample, which second reagent is affixed to or interspersed with the scaffold of (B) or which binds covalently or non covalently to the first capture reagent of (C);
(H) an indicator means which indicates that the sample has been transported along the substrate or support and confirms that the analyte of interest has been captured;
comprising selecting a liquid deposition device and depositing each or any of the scaffold, first capture reagent, second capture reagent, and indicator with said liquid deposition device in a regular and predetermined pattern.

17. The method of claim 16 wherein the liquid deposition device is an inkjet printer.

18. The method of claim 16 wherein
(A) the substrate or solid support is selected from glass, nylon, paper, nitrocellulose, and plastic;
(B) the scaffold or polymer is selected from nucleic acid, peptide, carbohydrate, and protein; and
(C) the first capture reagent is selected from antibody, antigen, peptide, nucleic acid, protein, ligand, carbohydrate, metal, fat, oil, and organic compound.

19. The method of claim 18 wherein the scaffold or polymer is nucleic acid and the first capture reagent comprises complementary nucleic acid, optionally attached to an antibody.

20. A process for application of a liquid reagent to a printable surface for capture of an analyte in a sample, said process utilizing an inkjet printer, comprising loading the liquid reagent into a printer ink cartridge for said inkjet printer and printing the reagent in a regular and predetermined pattern on the printable surface.
